(19) United States
(12) Patent Application Publication
(10) Pub. No.: US 2024/0076728 A1
(43) Pub. Date: Mar. 7, 2024

(54) NOVEL METHOD OF COMBINED MOLECULAR CLAMPING AND ALLELE SPECIFIC qPCR TECHNOLOGY FOR KRAS G12C MUTATION DETECTION

(71) Applicants: Qing Sun, Fairfield, CA (US); Hui Ren, South San Francisco, CA (US); Michael Y Sha, Castro Valley, CA (US); Michael J Powell, Alamo, CA (US); Aiguo Zhang, San Ramon, CA (US)

(72) Inventors: Qing Sun, Fairfield, CA (US); Hui Ren, South San Francisco, CA (US); Michael Y Sha, Castro Valley, CA (US); Michael J Powell, Alamo, CA (US); Aiguo Zhang, San Ramon, CA (US)

(21) Appl. No.: 18/347,492

(22) Filed: Jul. 5, 2023

Related U.S. Application Data
(63) Continuation of application No. 17/386,451, filed on Jul. 27, 2021, now abandoned.
(60) Provisional application No. 63/056,755, filed on Jul. 27, 2020.

(51) Int. Cl. C12Q 1/6827

(52) U.S. Cl. CPC C12Q 1/6827

(57) ABSTRACT

The invention provides a method for detecting KRAS mutations at one or more of codons, said method comprising the steps of: (a) extracting DNA from a biological sample; (b) assaying the DNA via PCR for KRAS mutations at one or more of codons with at least one set of oligonucleotides, wherein the at least one set of oligonucleotides comprises an allele specific forward primer, a reverse primer, a probe and a xenonucleic acid clamp to block amplification of wild type DNA. The xenonucleic acid clamps have aza-aza, thio-aza and oxy-aza chemical functionality.

This application is a continuation of pending application Ser. No. 17/386,451 filed Jul. 27, 2021; the contents of which are incorporated by reference herein. This application also claims the priority benefit under 35 U.S.C. section 119 from Provisional ApplicationNo. 63/056,755 filed Jul. 27, 2020 entitled “A Novel Method Of Combined Molecular and Allele Specific qPCR Technology For KRAS G12C Mutation Detection” which is in its entirety herein incorporated by reference.

### FIELD OF THE INVENTION

The present invention relates generally to oligonucleotides and methods for detecting KRAS and other mutations in patient samples. More specifically, the present invention relates to primers and PCR assays that are capable of detecting KRAS codon mutations with high specificity and sensitivity.

### BACKGROUND OF THE INVENTION

The family of Ras genes encodes small GTPases that are involved in cellular signal transduction. Mutations in Ras genes can permanently activate the genes and cause inappropriate transmission inside the cell in the absence of extracellular signals. Because the signals result in cell growth and division, dysregulated Ras signaling can ultimately lead to oncogenesis and cancer. The Ras genes encode the Ras superfamily of proteins, which includes the KRAS (Kirsten rat sarcoma viral oncogene homolog) protein, which is encoded by the KRAS gene.

KRAS gene mutations are common in pancreatic cancer, lung adenocarcinoma, colorectal cancer, gall bladder cancer, thyroid cancer, and bile duct cancer. The status of KRAS mutations have been reported as predictive markers of tumor response to epidermal growth factor receptor (EGFR) TKI-targeted therapies; accordingly, the mutational status of KRAS can provide important information prior to the prescription of TKI therapy.

The most common KRAS mutations occur in codons 12 and 13 of exon 2. Other more rarely occurring mutations have been seen in codons 59 and 61 of exon 3. KRAS mutations at codons 12, 13, or 61 have been found to cause Ras proteins to remain longer in their active form, resulting in an over-stimulation of the EGFR pathway; consequently, patients with KRAS mutations at codons 12, 13, or 61 do not respond well to TKI therapy. Further, mutations in KRAS codon 12 or 13 have been shown to be strong predictors of patient non-responsiveness to anti-EGFR monoclonal antibody therapies, such as ERBITUX® (cetuximab; ImClone Systems Inc., New York, N.Y., USA) and VECTIBIX® (panitumumab, Amgen, Thousand Oaks, Calif., USA) for the treatment of certain cancerous conditions, including metastatic colorectal cancer (mCRC) and lung cancer. Massarelli et al., KRAS Mutation is an Important Predictor of Resistance to Therapy with Epidermal Growth Factor Receptor Tyrosine Kinase Inhibitors in Non-Small Cell Lung Cancer, CLIN CANCER RES. 13(10):2890-2896 (2007); Amado et al., Wild-type KRAS is Required for Panitumumab Efficiency in Patients with Metastatic Colorectal Cancer, J. CLIN ONCOL 26(10):1626-1634 (2008); Van Cutsem et al., KRAS Status and Efficacy in the First-Line Treatment of Patients with Metastatic Colorectal Cancer (mCRC) Treated with FOLFIRI with or without Cetuximab: The CRYSTAL Experience, J CLIN ONCOL 26(15S): May 20 Supplement, Abstract 2 (2008); Baker et al., Evaluation of Tumor Gene Expression and KRAS Mutations in FFPE Tumor Tissue as Predictors of Response to Cetuximab in Metastatic Colorectal Cancer, J CLIN ONCOL 26(15S): May 20 Supplement, Abstract 3512 (2008); Van Zakowski et al., Reflex Testing of Lung Adenocarcinomas for EGFR and KRAS Mutations: The Memorial Sloan-Kettering Experience, J. CLIN ONCOL 26(15S):

### May 20 Supplement, Abstract 22031 (2008).

The role of activated KRAS in malignancy was observed over thirty years ago (e.g., see Santos et al., (1984) Science 223:661-664). Aberrant expression of KRas accounts for up to 20% of all cancers and oncogenic KRas mutations that stabilize GTP binding and lead to constitutive activation of KRas and downstream signaling have been reported in 25-30% of lung adenocarcinomas. (e.g., see Samatar and Poulikakos (2014) Nat Rev Drug Disc 13(12): 928-942 doi: 10.1038/nrd 428). Single nucleotide substitutions that result in missense mutations at codons 12 and 13 of the KRas primary amino acid sequence comprise approximately 40% of these KRas driver mutations in lung adenocarcinoma, with a G12C transversion being the most common activating mutation (e.g., see Dogan et al., (2012) Clin Cancer Res. 18(22):6169-6177, published online 2012 Sep. 26. doi: 10.1158/1078-0432.CCR-11-3265).

The well-known role of KRAS in malignancy and the discovery of these frequent mutations in KRAS in various tumor types made KRAS a highly attractable target of the pharmaceutical industry for cancer therapy. Notwithstanding thirty years of large scale discovery efforts to develop inhibitors of KRas for treating cancer, no KRAS inhibitor has demonstrated sufficient safety and/or efficacy to obtain regulatory approval (e.g., see McCormick (2015) Clin Cancer Res. 21 (8):1797-1801).

Additionally, it is known that allele-specific amplification of nucleic acids allows for simultaneous amplification and analysis of the target sequence. Allele-specific amplification is commonly used when the target nucleic acid has one or more variations (polymorphisms) in its sequence. Nucleic acid polymorphisms are used in DNA profile analysis (forensics, paternity testing, tissue typing for organ transplants), genetic mapping, distinguishing between pathogenic strains of microorganisms as well as detection of rare mutations, such as those occurring in cancer cells, existing in the background of cells with normal DNA.

In a successful allele-specific amplification, the desired variant of the target nucleic acid is amplified, while the other variants are not, at least not to a detectable level. A typical allele-specific amplification assay involves a polymerase chain reaction (PCR) with at least one allele-specific primer designed such that primer extension occurs only when the primer forms a hybrid with the desired variant of the target sequence. When the primer hybridizes to an undesired variant of the target sequence, primer extension is inhibited.

Many ways of enhancing allele-specificity of primers have been proposed. However, for many clinically-relevant nucleic acid targets lack of specificity of PCR remains a problem. Therefore radically novel approaches to design of allele-specific primers are necessary.

KRAS G12C is a common mutation which present in approximately 13% of lung adenocarcinoma, 3% of colorectal cancer and 2% of other solid tumors. Amgen has developed a promising KRAS G12C inhibitor which currently is enrolling for phase 1 and phase 2 clinical trials. Therefore, the invention provides a robust companion diagnostic kit for the detection of KRAS G12C mutation using qPCR assay.

### BRIEF DESCRIPTION OF THE FIGURES

FIG. 1 illustrates the mechanism of the XNA clamping process.

FIG. 1A illustrates the differences between DNA and XNA.

FIG. 2A illustrates that wild type DNA (without KRAS G12C mutation) produced a weak signal with Ct ˜47 without the clamping of XNA.

FIG. 2B shows that no amplification (Ct>50) has been observed when 250 nM XNA was used.

FIG. 3A is the amplification plot of reference sample diluted to 5% VAF.

FIG. 3B shows the amplification plot of reference sample diluted to 2.5% VAF.

FIG. 3C illustrates the amplification plot of reference sample diluted to 1% VAF.

FIG. 3D is the amplification plot of reference sample diluted to 0.5% VAF.

FIG. 3E shows the amplification plot of reference sample diluted to 0.25% VAF.

FIG. 3F illustrates the amplification plot of reference sample diluted to 0.1% VAF.

FIG. 4A shows that for known reference wildtype gDNA, the assay specificity is 100%. and there were no false positive calls for 5 ng of gDNA per reaction.

FIG. 4B illustrates that the allele specific forward primer binds to KRAS G12C mutant, but not KRAS G12A.

FIG. 4C shows that the allele specific forward primer binds to KRAS G12C mutant, but not KRAS G12D.

FIG. 4D describes that the allele specific forward primer binds to KRAS G12C mutant, but not KRAS G12R.

FIG. 4E shows that the allele specific forward primer binds to KRAS G12C mutant, but not KRAS G12S.

FIG. 4F illustrates that the allele specific forward primer binds to KRAS G12C mutant, but not KRAS G12V.

FIG. 4G describes that the allele specific forward primer binds to KRAS G12C mutant, but not KRAS G13D.

FIG. 4H shows that the allele specific forward primer binds to KRAS G12C mutant, but not BRAF V600E.

### SUMMARY OF THE INVENTION

The invention provides a method of detecting KRAS mutations at one or more of codons; said method comprising: (a) providing a biological sample; (b) isolating DNA from said biological sample; said DNA including said KRAS mutations; (c) providing a first primer primer probe which is allele specific and a second primer probe; wherein said first and second primer probes are targeted to said KRAS mutations and wherein said primer probes allow formation of a PCR process product; (d) providing a target specific xenonucleic acid clamp oligomer probe specific for a wildtype polynucleotide sequence; so that during the qPCR process only mutant templates are amplified; (e) admixing the primer probes and the xenonucleic clamping probe with the target nucleic acid sample; (f) performing a PCR amplification process in a reaction solution under hybridization conditions thereby generating multiple amplicons; and (g) detecting said amplicons.

The invention also provides a method of detecting KRAS mutations at one or more of codons, said method comprising the steps of: (a) extracting DNA from a biological sample; (b) assaying the DNA via PCR for KRAS mutations at one or more of codons with at least one set of oligonucleotides, wherein the at least one set of oligonucleotides comprises an allele specific forward primer, a reverse primer, a probe and a xenonucleic acid clamp to block amplification of wild type DNA

In a further aspect of the invention, there is provided a PCR kit for detecting KRAS mutations, comprising PCR reagent mixes for detection of KRAS mutations at one or more of codons, comprising the KRAS oligonucleotide probes of the present invention; Taq polymerase; and instructions for use.

The invention also provides a method for detecting one or more KRAS mutations selected from Gly12Ser, Gly12Arg, Gly12Cys, Gly12Asp, Gly12Ala, Gly12Val, Gly13Asp, Gln61His and Gln61Leu in a test sample comprising nucleic acid, wherein said method comprises subjecting the sample to amplification with a mixture comprising one or more primers and a xenonucleic acid clamp to block amplification of the wild type nucleic acid.

### DETAILED DESCRIPTION OF THE INVENTION

As used herein, the term “oligonucleotide” refers to a molecule comprising two or more deoxyribonucleotides, ribonucleotides, and/or nucleotide analogs, the latter including nucleic acid analogs, such as isoguanosine, isocytosine, inosine, or deoxyinosine. The length of the oligonucleotide will vary depending on the function of the oligonucleotide. The oligonucleotide may be generated in any manner, including chemical synthesis, DNA replication, reverse transcription, PCR, or a combination thereof. As used herein, the term “oligonucleotide” is meant to encompass primers (both forward and reverse primers) and detection probes.

As used herein, the term “primer” refers to an oligonucleotide which, whether purified from a nucleic acid restriction digest or produced synthetically, is capable of acting as a point of initiation of nucleic acid synthesis when placed under conditions in which synthesis of a primer extension product which is complementary to a nucleic acid strand is induced, i.e., in the presence of nucleotides and an agent for polymerization such as DNA polymerase, reverse transcriptase or the like, and at a suitable temperature and pH. The primer is preferably single stranded for maximum efficiency, but may alternatively be double stranded. If double stranded, the primer is first treated to separate its strands before being used to prepare extension products. The primer must be sufficiently long to prime the synthesis of extension products in the presence of the agents for polymerization. The exact lengths of the primers will depend on many factors, including temperature and the source of primer. For example, depending on the complexity of the target sequence, a primer typically contains 15 to 25 or more nucleotides, although it can contain fewer nucleotides. Short primer molecules generally require cooler temperatures to form sufficiently stable hybrid complexes with a template.

The term “forward primer” refers to a primer that forms an extension product by binding in the 5′ to 3′ direction to the 3′ end of a strand of a denatured DNA analyte.

The term “reverse primer” refers to a primer that forms an extension product by binding in the 3′ to 5′ direction to the 5′ end of a strand of a denatured DNA analyte.

The term “amplicon” refers to the amplification product of a nucleic acid extension assay, such as PCR.

As used herein, the term “probe” or “detection probe” refers to an oligonucleotide that forms a hybrid structure with a target sequence contained in a molecule (i.e., a “target molecule”) in a sample undergoing analysis, due to complementarity of at least one sequence in the probe with the target sequence.

As used herein, the term “melting temperature” (Tm) in relation to an oligonucleotide is defined as the temperature at which 50% of the DNA forms a stable double-helix and the other 50% has been separated into single stranded molecules. As known to those of skill in the art, PCR annealing temperature is typically a few degrees less than the Tm, the latter of which is calculated based on oligo and salt concentrations in the reaction.

The term “biological sample” as used herein is meant to include both human and animal species.

The term “gene” refers to a particular nucleic acid sequence within a DNA molecule that occupies a precise locus on a chromosome and is capable of self-replication by coding for a specific polypeptide chain. The term “genome” refers to a complete set of genes in the chromosomes of each cell of a specific organism.

The term “target” refers to a molecule, gene, or genome containing a nucleotide, nucleic acid sequence, or sequence segment that is intended to be characterized by way of detection, amplification, or quantification.

The term “single nucleotide polymorphism” or “SNP” refers to single point variations in genomic DNA or tumor-associated DNA. It is to be understood that within the context of the present invention, the terms “mutation” and “point mutation” are meant to include and/or refer to SNPs.

As used herein, the term “KRAS” refers to the human cellular homolog of a transforming gene isolated from the Kirsten rat sarcoma virus, as defined by NCBI's OMIM database entry 190070.

A sample that comprises “both wild type copies of the KRAS gene and mutant copies of the KRAS gene” and grammatical equivalents thereof, refers to a sample that contains multiple DNA molecules of the same genomic locus, where the sample contains both wild type copies of the genomic locus (which copies contain the wild type allele of the locus) and mutant copies of the same locus (which copies contain the mutant allele of the locus). In this context, the term “copies” is not intended to mean that the sequences were copied from one another. Rather, the term “copies” in intended to indicate that the sequences are of the same locus in different cells or individuals.

As used herein the term “nucleotide sequence” refers to a contiguous sequence of nucleotides in a nucleic acid. As would be readily apparent, number of nucleotides in a nucleotide sequence may vary greatly. In particular embodiments, a nucleotide sequence (e.g., of an oligonucleotide) may be of a length that is sufficient for hybridization to a complementary nucleotide sequence in another nucleic acid. In these embodiments, a nucleotide sequence may be in the range of at least 10 to 50 nucleotides, e.g., 12 to 20 nucleotides in length, although lengths outside of these ranges may be employed in many circumstances.

The invention provides a method of detecting KRAS mutations at one or more of codons; said method comprising: (a) providing a biological sample; (b) isolating DNA from said biological sample; said DNA including said KRAS mutations; (c) providing a first primer primer probe which is allele specific and a second primer probe; wherein said first and second primer probes are targeted to said KRAS mutations and wherein said primer probes allow formation of a PCR process product; (d) providing a target specific xenonucleic acid (XNA) clamp oligomer probe specific for a wildtype polynucleotide sequence; so that during the qPCR process only mutant templates are amplified; (e) admixing the primer probes and the xenonucleic acid clamping probe with the target nucleic acid sample; (f) performing a PCR amplification process in a reaction solution under hybridization conditions thereby generating multiple amplicons; and (g) detecting said amplicons.

The invention also provides a method of detecting KRAS mutations at one or more of codons, said method comprising the steps of: (a) extracting DNA from a biological sample; (b) assaying the DNA via PCR for KRAS mutations at one or more of codons with at least one set of oligonucleotides, wherein the at least one set of oligonucleotides comprises an allele specific forward primer, a reverse primer, a probe and a xenonucleic acid clamp to block amplification of wild type DNA

Utilizing xeno-nucleic acid (XNA) clamping probes in the PCR mediated amplification of DNA templates, only target genetic material that has a mutation or variation, e.g. single nucleotide polymorphism (SNP), gene deletion or insertion and/or translocation or truncation is amplified in the oligonucleotide primer directed polymerase chain reaction (qPCR).

The XNA probe clamping sequences are designed to bind specifically by Watson-Crick base pairing to abundant wild-type sequences in the DNA templates derived from the biological sample of interest. The presence of the XNA probes in the PCR primer mix employed for the target amplification reaction causes inhibition of the polymerase mediated amplification of wild-type templates but does not impede the amplification of mutant template sequences.

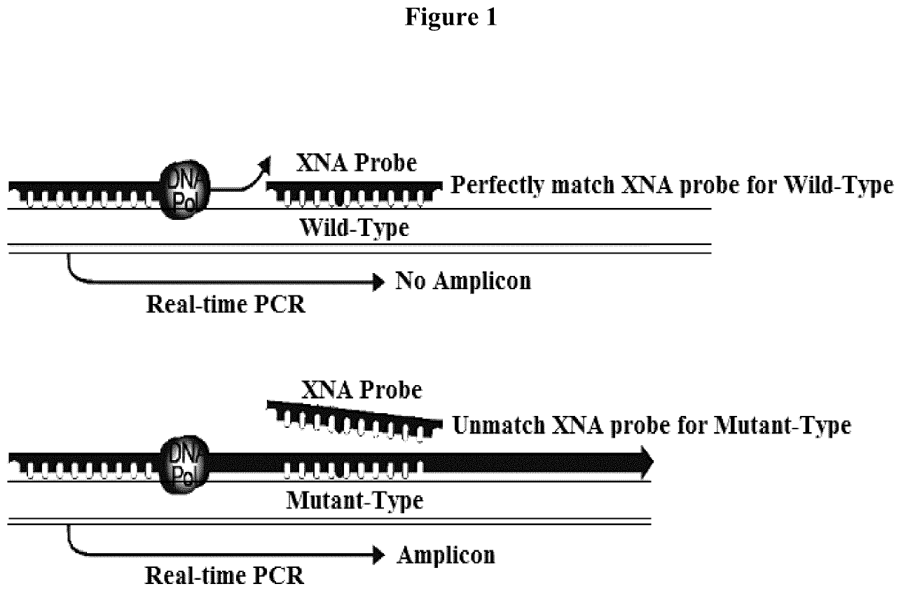

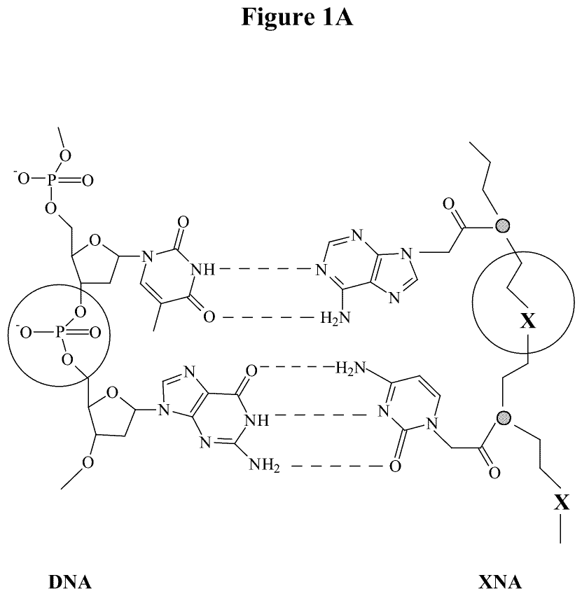

The mechanism of the XNA clamping process is depicted in FIG. 1. As shown in FIG. 1, the modified DNA oligo probe binds or clamps to wild type DNA and blocks further wild type amplification. This probe or XNA “clamp” does not bind to mutated DNA, allowing it to be amplified and detected.

The suppression of wild-type (wt) template amplification and amplification of only mutant templates is achieved because there is a differential melting temperature (Tm) between the XNA clamp bound to mutant templates vs wild type templates:
Tm(XNA mutant template)<<Tm (XNA wt template)

The Tm differential is as much as 15-20° C. for the XNA clamp probes. So that during the PCR process only mutant templates are amplified.

For purposes of illustration, FIG. 1A illustrates the differences between DNA and XNA.

Applicant has developed a multitude of XNA chemistry and multiple applications of XNA in molecular testing including, PCR-Clamping, in-situ detection of gene mutations and targeted CRISPR/Cas9 gene-editing and detection. Applicant's XNA chemistry is unique in that a single nucleotide change in the target sequence can lead to a melting temperature differential of as much as 15-200° C. For natural DNA the Tm differential for such a change is only 5-70° C.

Representative examples are shown below:

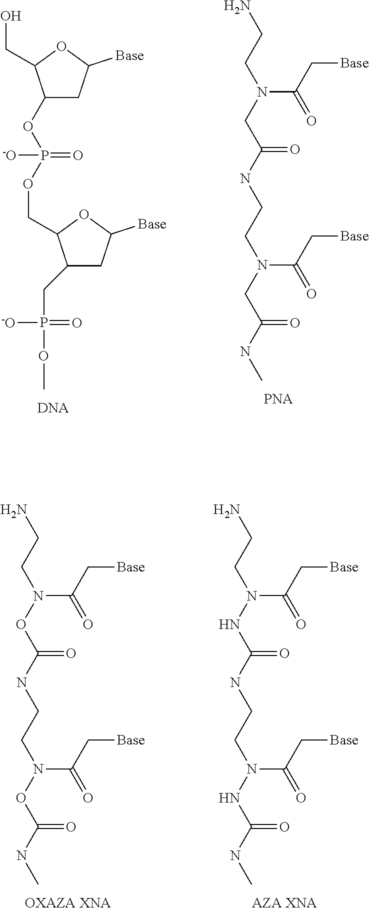

where base is selected from the group consisting of adenine, cytosine, guanine, thymine and uracil.

The xenonucleic acid clamps have aza-aza, thio-aza and oxy-aza chemical functionality and selected from the group consisting of the following chemical structures;

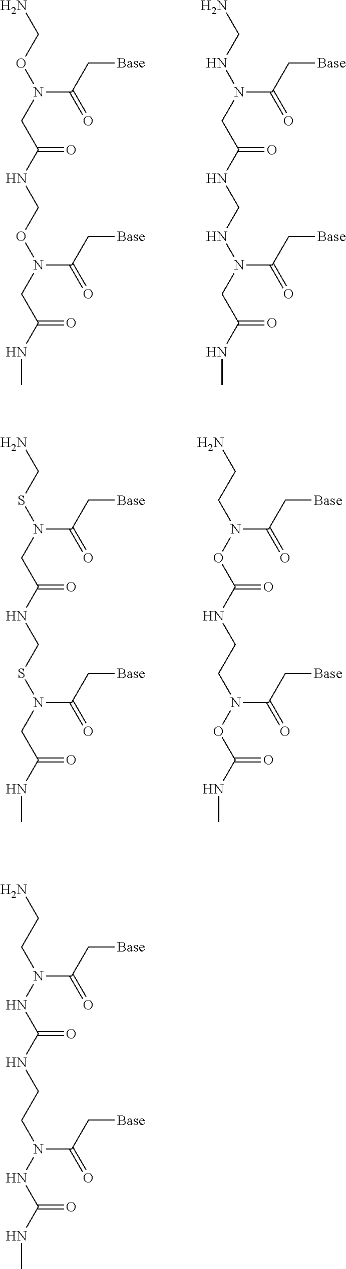

where base is selected from the group consisting of adenine, cytosine, guanine, thymine and uracil.

The XNA monomers are synthesized as shown in the following schemes:

### Synthesis of Xenonucleic Acid (XNA) Monomers

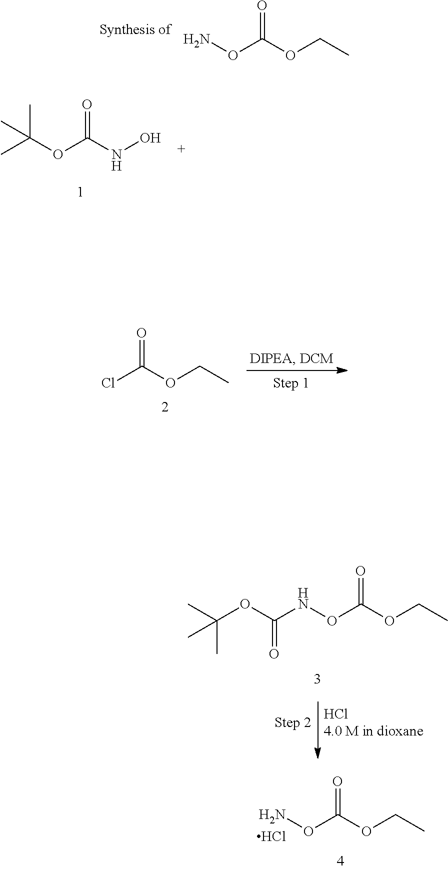

### Aza-XNA Monomer Synthesis

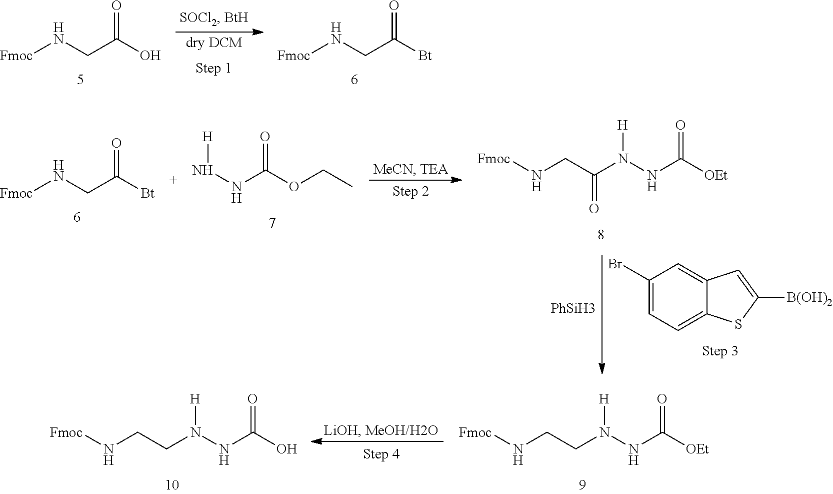

### Synthesis of Oxaza-XNA Monomer

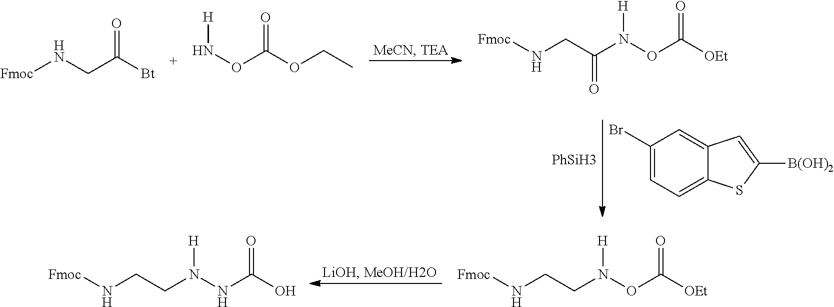

### Attachment of Protected Nucleic Acid Bases and Solid Phase Synthesis of XNA Oligomers

### Benzothiazole-2-Sulfonyl-(Bts) Route to XNA Monomer Synthesis

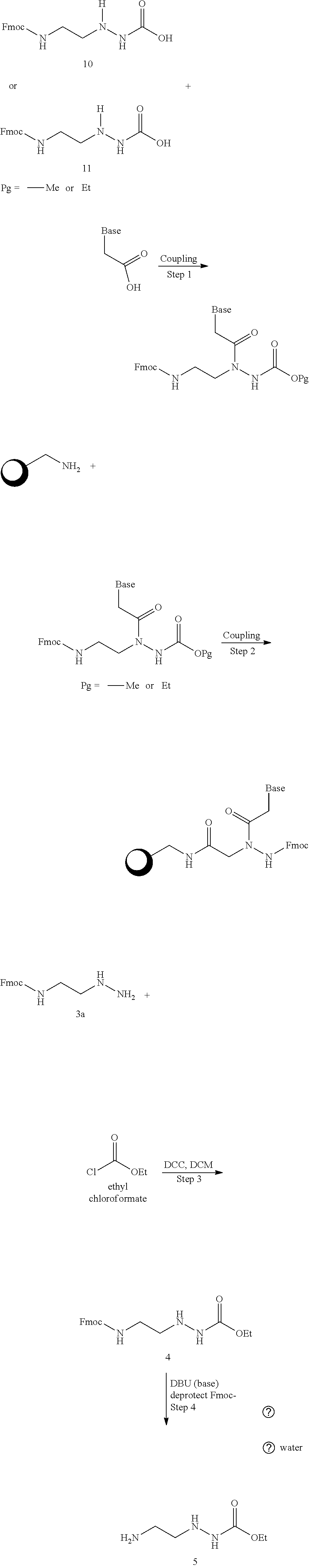

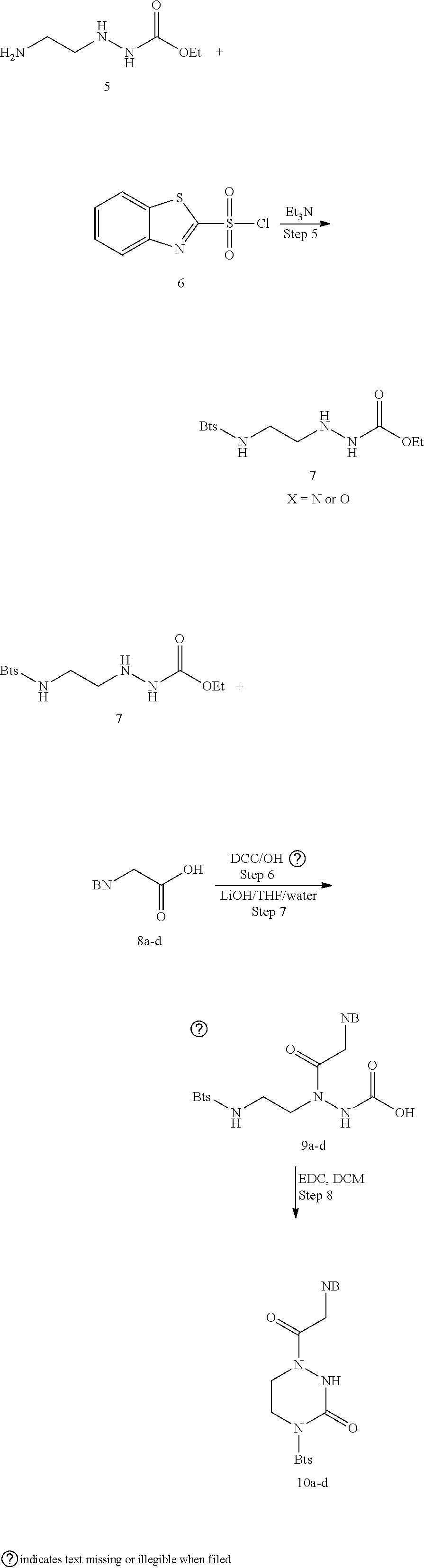

We could also introduce CDI (carbonyldiimidazole chemistry; by doing that we may skip Step 7 in above and can get to the final cyclized monomer.

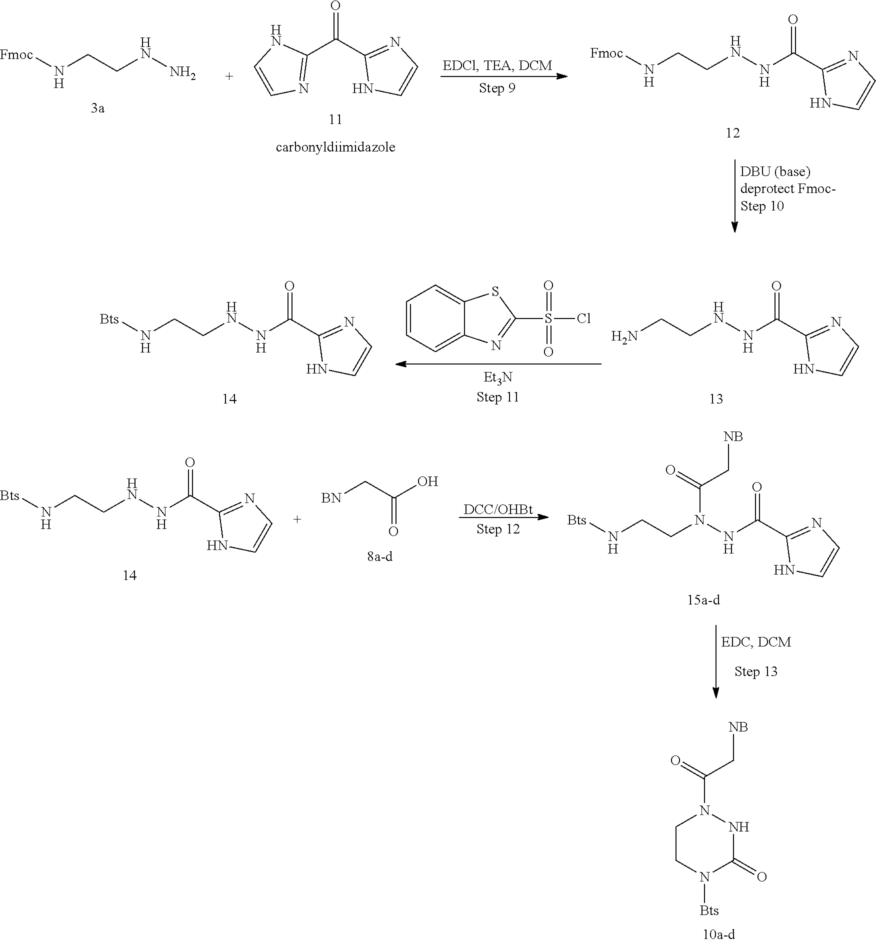

Another aza-aza compound having the structure below is made by the following synthetic steps:

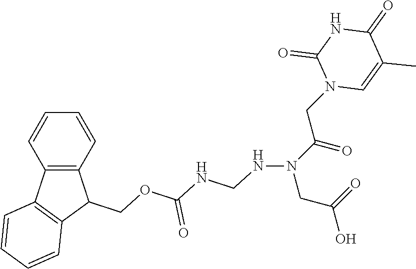 [SMILES: Cc1cn(CC(=O)N(CC(=O)O)NCNC(=O)OCC2c3ccccc3-c3ccccc32)c(=O)[nH]c1=O]

Thymine Aza-Aza Analog

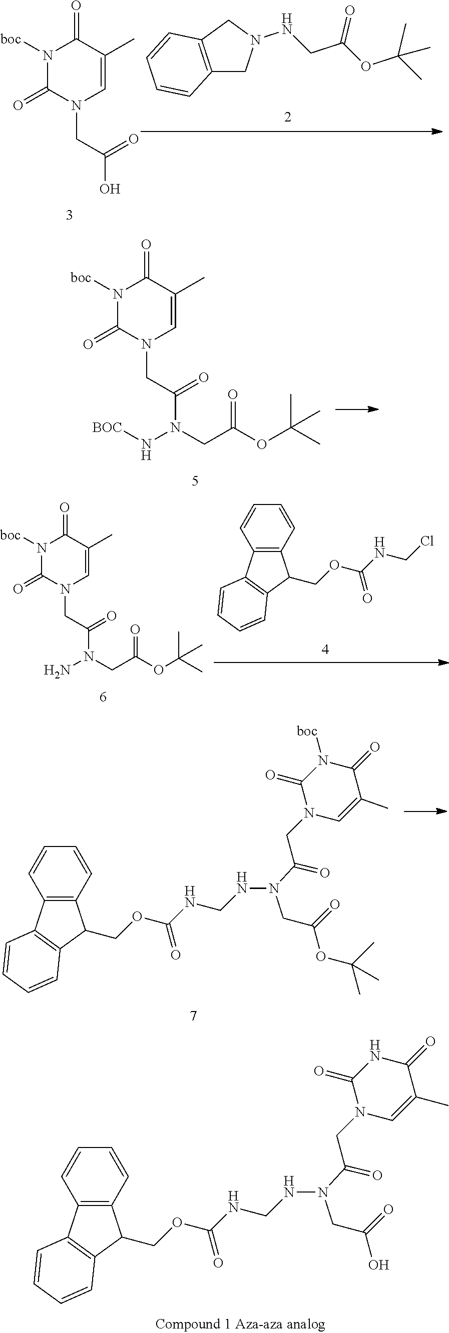

Another compound of the invention is a thio-aza compound having the following chemical structure and made by the synthetic scheme below:

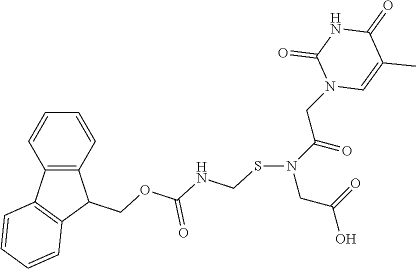 [SMILES: Cc1cn(CC(=O)N(CC(=O)O)SCNC(=O)OCC2c3ccccc3-c3ccccc32)c(=O)[nH]c1=O]

### Thymine Sulfa-Aza Monomer

The synthesis of the above compound is as follows:

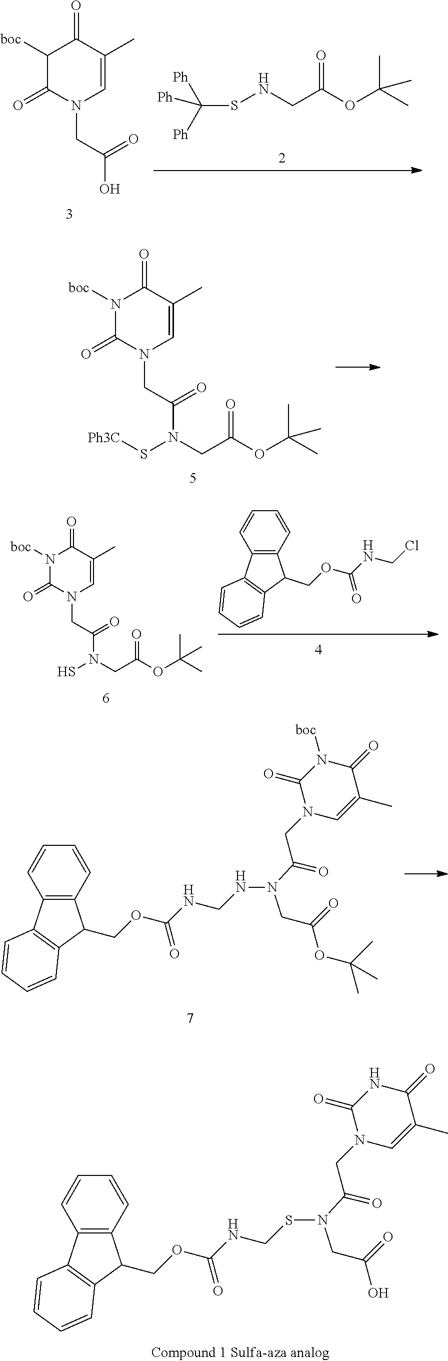

The synthetic methodology of the invention is used to synthesize the following aza-aza and oxy-aza compounds:

### Aza-Aza

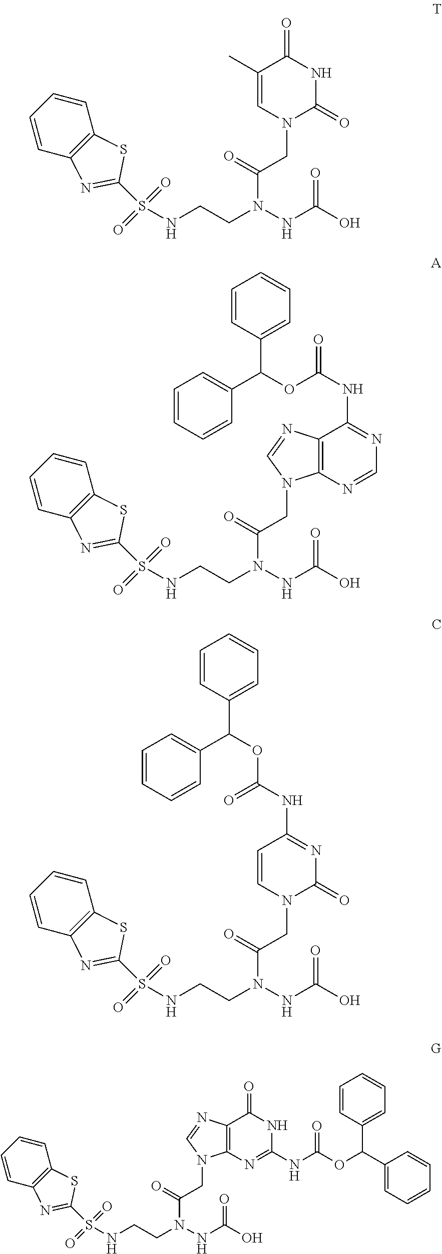 [SMILES: Cc1cn(CC(=O)N(CCNS(=O)(=O)c2nc3ccccc3s2)NC(=O)O)c(=O)[nH]c1=O ; O=C(O)NN(CCNS(=O)(=O)c1nc2ccccc2s1)C(=O)Cn1cnc2c(NC(=O)OC(c3ccccc3)c3ccccc3)ncnc21 ; O=C(O)NN(CCNS(=O)(=O)c1nc2ccccc2s1)C(=O)Cn1ccc(NC(=O)OC(c2ccccc2)c2ccccc2)nc1=O ; O=C(O)NN(CCNS(=O)(=O)c1nc2ccccc2s1)C(=O)Cn1cnc2c(=O)[nH]c(NC(=O)OC(c3ccccc3)c3ccccc3)nc21]

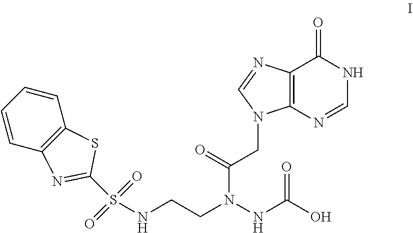 [SMILES: O=C(O)NN(CCNS(=O)(=O)c1nc2ccccc2s1)C(=O)Cn1cnc2c(=O)[nH]cnc21]

### Oxy Aza

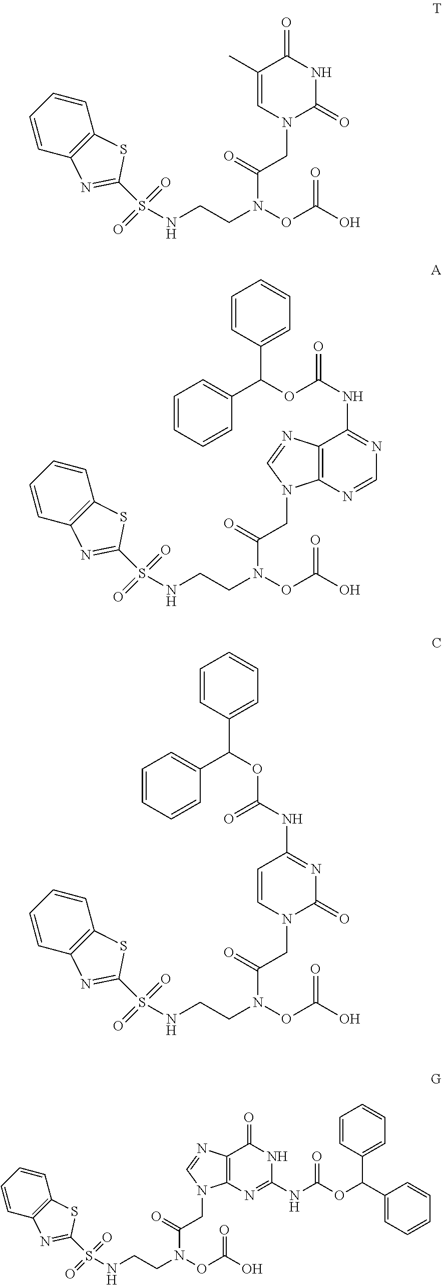 [SMILES: Cc1cn(CC(=O)N(CCNS(=O)(=O)c2nc3ccccc3s2)OC(=O)O)c(=O)[nH]c1=O ; O=C(O)ON(CCNS(=O)(=O)c1nc2ccccc2s1)C(=O)Cn1cnc2c(NC(=O)OC(c3ccccc3)c3ccccc3)ncnc21 ; O=C(O)ON(CCNS(=O)(=O)c1nc2ccccc2s1)C(=O)Cn1ccc(NC(=O)OC(c2ccccc2)c2ccccc2)nc1=O ; O=C(O)ON(CCNS(=O)(=O)c1nc2ccccc2s1)C(=O)Cn1cnc2c(=O)[nH]c(NC(=O)OC(c3ccccc3)c3ccccc3)nc21]

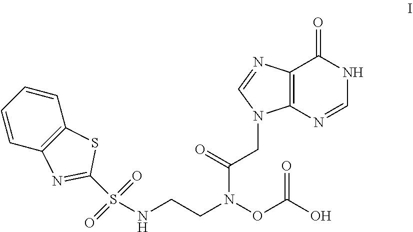 [SMILES: O=C(O)ON(CCNS(=O)(=O)c1nc2ccccc2s1)C(=O)Cn1cnc2c(=O)[nH]cnc21]

Aza-Aza

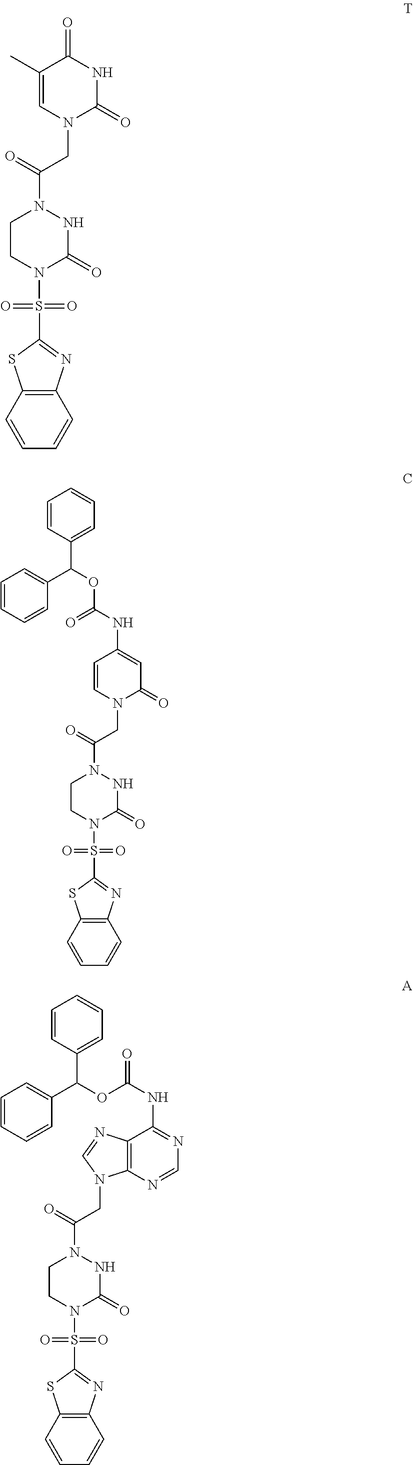

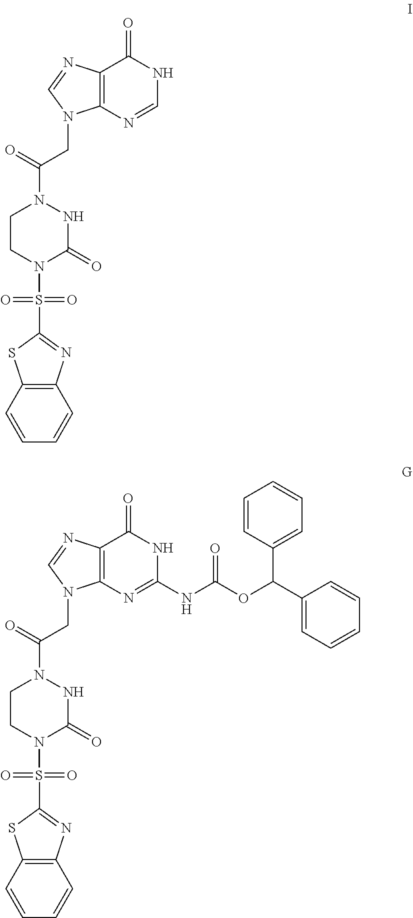 [SMILES: O=C(Cn1cnc2c(=O)[nH]cnc21)N1CCN(S(=O)(=O)c2nc3ccccc3s2)C(=O)N1 ; O=C(Nc1nc2c(ncn2CC(=O)N2CCN(S(=O)(=O)c3nc4ccccc4s3)C(=O)N2)c(=O)[nH]1)OC(c1ccccc1)c1ccccc1]

### Oxy Aza

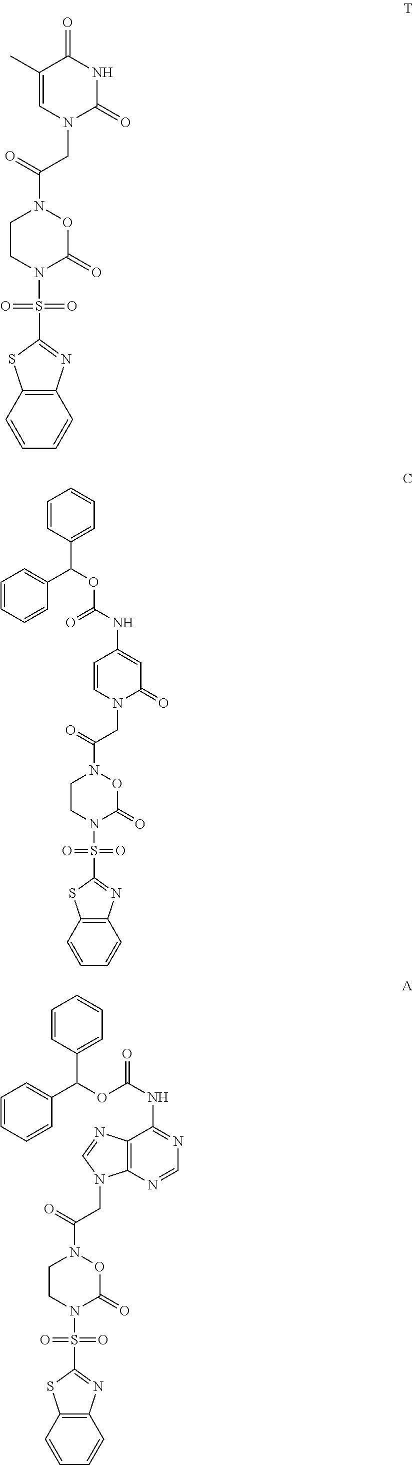

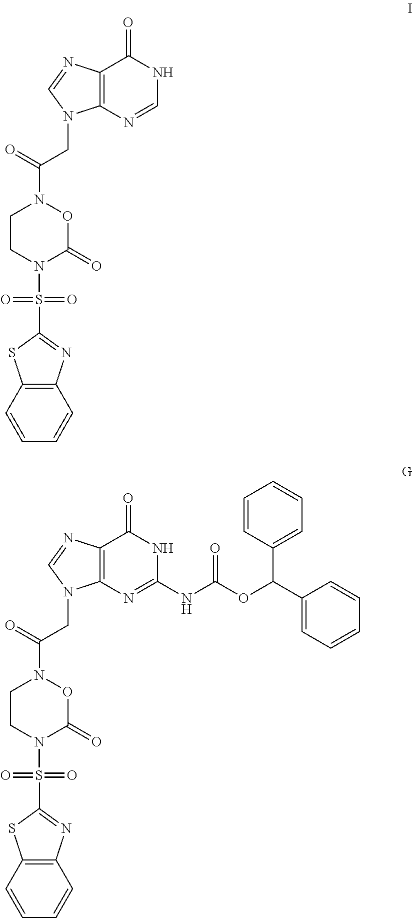 [SMILES: O=C(Cn1cnc2c(=O)[nH]cnc21)N1CCN(S(=O)(=O)c2nc3ccccc3s2)C(=O)O1 ; O=C(Nc1nc2c(ncn2CC(=O)N2CCN(S(=O)(=O)c3nc4ccccc4s3)C(=O)O2)c(=O)[nH]1)OC(c1ccccc1)c1ccccc1]

The synthetic scheme below is used to make alternative isomeric forms of aza-XNA isomer:

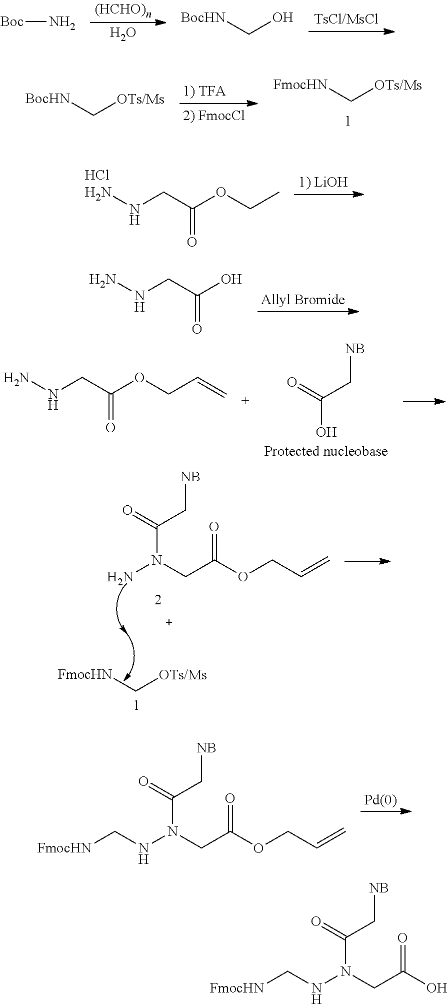

The methods disclosed herein can be used to analyze nucleic acids of samples. The term “sample” as described herein can include bodily fluids (including, but not limited to, blood, urine, feces, serum, lymph, saliva, anal and vaginal secretions, perspiration, peritoneal fluid, pleural fluid, effusions, ascites, and purulent secretions, lavage fluids, drained fluids, brush cytology specimens, biopsy tissue (e.g., tumor samples), explanted medical devices, infected catheters, pus, biofilms and semen) of virtually any organism, with mammalian samples, particularly human samples.

Amplification primers useful in the embodiments disclosed herein are preferably between 10 and 45 nucleotides in length. For example, the primers can be at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, or more nucleotides in length. Primers can be provided in any suitable form, included bound to a solid support, liquid, and lyophilized, for example. In some embodiments, the primers and/or probes include oligonucleotides that hybridize to a reference nucleic acid sequence over the entire length of the oligonucleotide sequence. Such sequences can be referred to as “fully complementary” with respect to each other. Where an oligonucleotide is referred to as “substantially complementary” with respect to a nucleic acid sequence herein, the two sequences can be fully complementary, or they may form mismatches upon hybridization, but retain the ability to hybridize under stringent conditions or standard PCR conditions as discussed below. As used herein, the term “standard PCR conditions” include, for example, any of the PCR conditions disclosed herein, or known in the art, as described in, for example, PCR 1: A Practical Approach, M. J. McPherson, P. Quirke, and G. R. Taylor, Ed., (c) 2001, Oxford University Press, Oxford, England, and PCR Protocols: Current Methods and Applications, B. White, Ed., (c) 1993, Humana Press, Totowa, NJ. The amplification primers can be substantially complementary to their annealing region, comprising the specific mutant/variant target sequence(s) or the wild type target sequence(s). Accordingly, substantially complementary sequences can refer to sequences ranging in percent identity from 100, 99, 98, 97, 96, 95, 94, 93, 92, 91, 90, 89, 85, 80, 75 or less, or any number in between, compared to the reference sequence. Conditions for enhancing the stringency of amplification reactions and suitable in the embodiments disclosed herein, are well-known to those in the art. A discussion of PCR conditions, and stringency of PCR, can be found, for example in Roux, K. “Optimization and Troubleshooting in PCR,” in Pcr Primer: A Laboratory Manual, Diffenbach, Ed. © 1995, Cold Spring Harbor Laboratory Press, Cold Spring Harbor, NY; and Datta, et al. (2003) Nucl. Acids Res. 31(19):5590-5597.

Activating mutations in KRAS are among the most common mutations found in a variety of cancers, and have long been recognized as a prominent tumor driver. KRAS mutations, including G12C, activate KRAS by interfering with GTPase-activating protein-mediated GTP hydrolysis, leading to signaling incompetent KRAS-GDP complexes in favor of KRAS-GTP. KRAS G12C is a single point mutation with a glycine-to-cysteine substitution at codon 12. KRAS G12C is present in approximately 13% of lung adenocarcinoma, 3% of colorectal cancer and 2% of other solid tumors.

KRAS was once believed to be undruggable. Attempts by biotech researchers to inhibit KRAS directly used to be challenging. Recently, however, it has been shown that the mutant cysteine of KRASG12C is located adjacent to a narrow pocket in the inactive GDP-bound form, making it susceptible to targeting. Encouragingly, Amgen has developed a promising KRAS G12C inhibitor which currently is enrolling for phase 1 and phase 2 clinical trials. Therefore, companion diagnostic (CDx) assays for KRAS G12C mutation detection are in great demand. The aim of this study is to develop a robust diagnostic kit for the detection of KRAS G12C mutation using qPCR assay.

This KRAS G12C mutation detection kit utilizes DiaCarta's proprietary QClamp® XNA technology for gene mutation detection in small number of mutated DNA population against the background of majority of normal (wild-type) DNA population. XNA is a synthetic DNA analog in which the phosphodiester backbone has been replaced by a units of DiaCarta's proprietary novel uncharged backbone chemistry. XNAs hybridize tightly to complementary DNA target sequences only if the sequence is a complete match. XNA oligomers are not recognized by DNA polymerases and cannot be utilized as primers in subsequent real-time PCR reactions. Binding of XNA to its target sequence blocks strand elongation by DNA polymerase. When there is a mutation in the target site, and therefore a mismatch, the XNA:DNA duplex is unstable, allowing strand elongation by DNA polymerase (See FIG. 1). Therefore, the high sensitivity of the assay is achieved.

The assay also incorporates KRAS G12C allele specific primer to ensure only KRAS G12C but not other SNPs is amplified in the qPCR assay. Allele-specific polymerase chain reaction (AS-PCR) is a PCR-based method which can be employed to detect the known SNPs. In this approach, the specific primers are designed to permit amplification by DNA polymerase only if the nucleotide at the 3′-end of the primer perfectly complements the base at the variant or wild-type sequences. It has been reported that introducing a single nucleotide artificial mismatch within the three bases closest to the 3′end (SNP site) improves AS-PCR performance.

The invention provides a novel XNA and allele specific primer-based real-time PCR assay for the detection of KRAS G12C mutations.

This KRAS G12C mutation detection kit utilizes DiaCarta's proprietary QClamp® XNA technology for gene mutation detection in small number of mutated DNA population against the background of majority of normal (wild-type) DNA population. It also combines allele-specific PCR technology using KRAS G12C allele specific primer to ensure only KRAS G12C but not other SNPs is amplified in the qPCR reaction.

The limit of detection of KRAS G12C is 0.25% VAF with 5ng total DNA input. This detection assay for KRAS G12C has no cross reactivity with KRAS G12A/D/S and BRAF V600E and very little reactivity with KRAS G12R/V and KRAS G13D) mutation.

This KRAS G12C mutation detection assay is robust with very high sensitivity and specificity. It can serve as a promising companion diagnostic kit for the detection of KRAS G12C mutation.

### EXAMPLES

### Materials and Methods

### Reference Standard Materials

Wild type genomic DNA (Bioline #BIO-35025) with no target mutations was used as negative control. The following genomic DNA reference materials carrying specific mutations were obtained from Horizon Discovery Group plc: KRAS G12A (Horizon #HD265), KRAS G12C (Horizon #HD269), KRAS G12D (Horizon #HD272), KRAS G12R (Horizon #HD287), KRAS G12S (Horizon #HD288), KRAS G12V (Horizon #HD289), KRAS G13D (Horizon #HD290), BRAF V600E (Horizon #HD238). These mutant reference DNA were prepared in wild type genomic DNA at 0.1-5% allelic frequency for specific tests.

### Example 1

### Primer, Probe and XNA Design

The high sensitivity of this KRAS G12C (COSM516) detection assay is achieved due to XNA clamping technology as illustrated in FIG. 1. KRAS G12C XNA is designed that bind to the selected wild-type sequences at the specific genetic loci in the target KRAS G12C gene. Primers and TaqMan hydrolysis probes were designed by Primer3 software (version 0.4.0) for the amplification of KRAS G12C. Allele specific forward primer was designed by introducing 2 artificial mismatch sites to specifically bind to KRAS G12C mutant variant. The human beta actin gene (ACTB) was selected as an internal control for the assay. All primers were synthesized by IDT (Integrated DNA Technology) and probes were ordered from BioSearch Inc. XNA oligomers were ordered from CPC Inc. The sequences of primers, probes and XNA were listed in Table 1.

TABLE 1
Primer, Probe, XNA sequences

| Name | | Sequence |
|---|---|---|
| KRAS G12C forward | SEQ ID NO: 1 | TGAATATAAACTTGTGGTAGTTGGAGCAT |
| primer(allele specific) | | |
| KRAS G12C reverse | SEQ ID NO: 2 | CCTCTATTGTTGGATCATATTCGTCCAC |
| primer | | |
| KRAS G12C probe | SEQ ID NO: 3 | TCTGAATTAGCTGTATCGTCAAGGCACTC |
| KRAS G12C XNA | SEQ ID NO: 4 | CTACGCCACCAGCTCCAACTACCA-O-D-Lys |
| ACTB forward primer | SEQ ID NO: 5 | TCTGCCTTACAGATCATGTTTGAG |
| ACTB reverse primer | SEQ ID NO: 6 | CCAGAGGCGTACAGGGATAG |
| ACTB probe | SEQ ID NO: 7 | CCATGTACGTTGCTATCCAGGCTGT |

### Example 2

### KRAS G12C Detection Assay

The assay consists of 10 μl of reaction volume including 5 ul of 2× buffer (Bioline #11060), 1 μl KRAS G12C primer/probe mix in 1×TE with final concentration of 400 nM primers and 200 nM probe, 1 μl of XNA final concentration at 0.25 μM, 1 μl ACTB primer/probe mix at the final concentration of 100 nM primers and 100 nM probe, and 2 μl of template (nuclease free water for non-template control or 5 ng DNA). Negative controls (NC, human wildtype gDNA) and positive controls (PC, include KRAS G12C mutant DNA) were included in each run. The thermocycling profile on QuantStudio 5 real-time PCR machine (Thermo Fisher) is as follows: 95° C. for 2 minutes followed by 50 cycles of 95° C. for 20 seconds, 70° C. for 40 seconds (for XNA binding), 66° C. for 30 seconds and 72° C. for 30 seconds. The complete assay consists of duplex qPCR reactions with XNAs to simultaneously detect both KRAS G12C mutation and ACTB. VIC/HTEX reporter was used to monitor KRAS G12C and CY5 reporter was used for ACTB.

### Example 3

### Data Analysis

The threshold of KRAS G12C and ACTB was set to 10000 and 5000 respectively. The mutational status of a sample was determined by calculating the Ct value between amplification reactions for a mutant allele assay and an internal control assay, as follows. Cq difference (ΔCq)=Mutation Assay Cq−Internal Control Assay Cq. The cut-off values were experimentally determined as its ΔCq value by testing at least 20 wildtype gDNA and/or cfDNA repeatedly during the verification of assay performance. If the ΔCq is <cut-off value, the mutation is detected as positive. If the ΔCq is >cut-off value, the mutation is not detected.

### Performance Parameters of the Assay

Performance parameters of the assay were established on reference DNA materials. Assay performance characteristics were verified with respect to limit of detection and cross-reactivity. The KRAS G12C reference DNA was diluted to 5%, 2.5%, 1%, 0.5%, 0.25% and 0.1% VAF respectively to test limit of detection. KRAS G12A, KRAS G12D, KRAS G12R, KRAS G12S, KRAS G12V, KRAS G13D and BRAF V600E mutant reference genomic DNA were diluted at 5% VAF to evaluate the cross-reactivity.

### Example 4

### Assay Feasibility

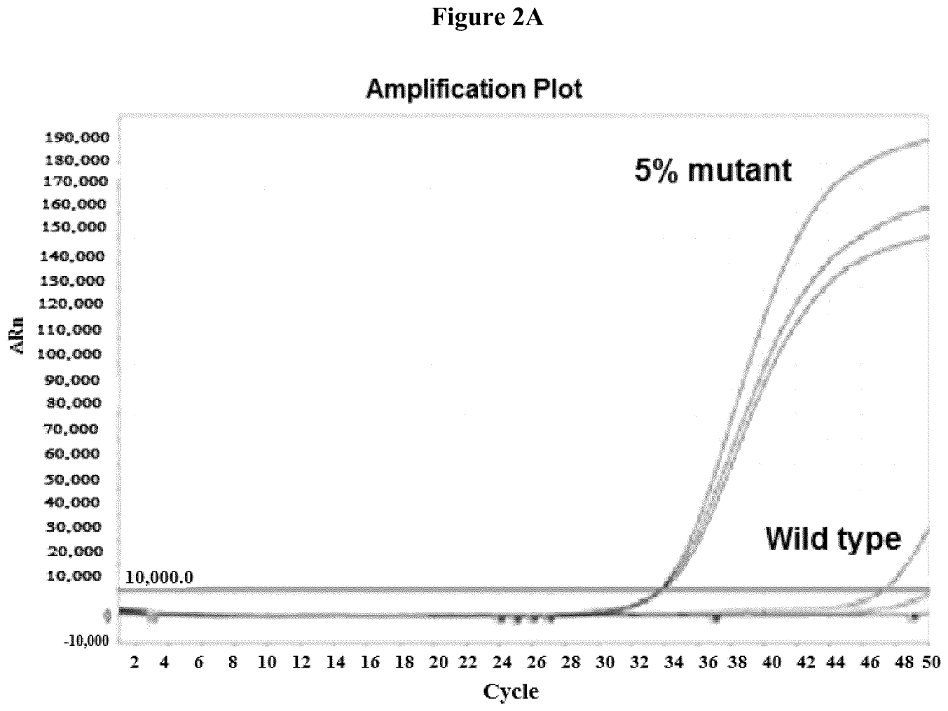

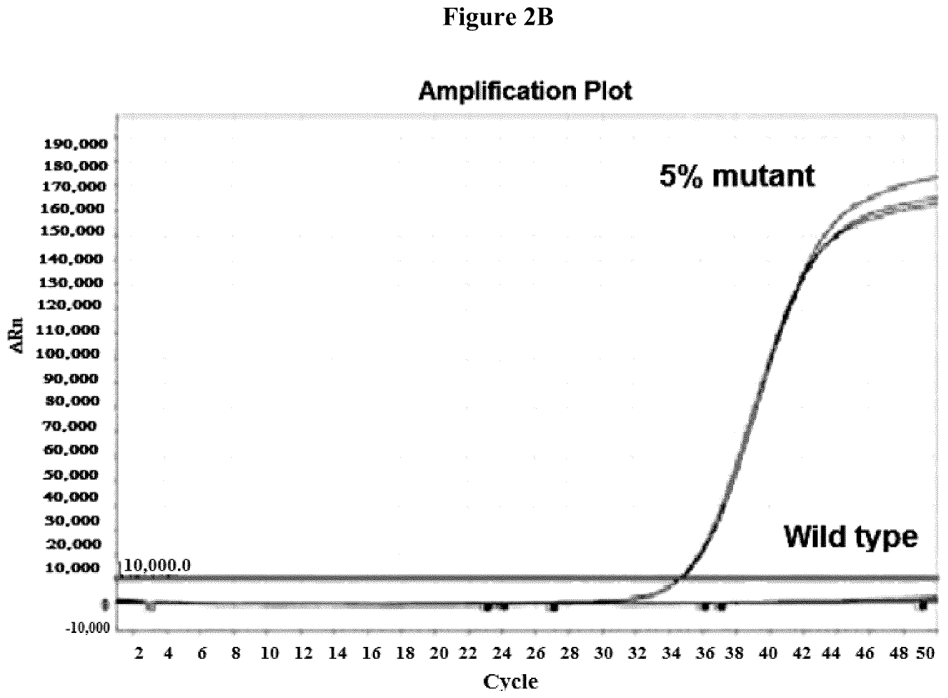

In order to demonstrate that XNA can effectively suppress wild-type allele amplification and thus enrich the mutations during qPCR, we compared XNA-based qPCR and qPCR without XNA. Wild type DNA (without KRAS G12C mutation) produced a weak signal with Ct ˜47 without the clamping of XNA (FIG. 2A), while no amplification (Ct>50) has been observed when 250 nM XNA was used (FIG. 2B). This demonstrates that XNA enables mutation detectable easily by blocking wildtype sequence amplification. FIG. 1 shows that XNA at 250 nM significantly blocks the amplification of wild type DNA.

### Example 5

### Analytical Sensitivity

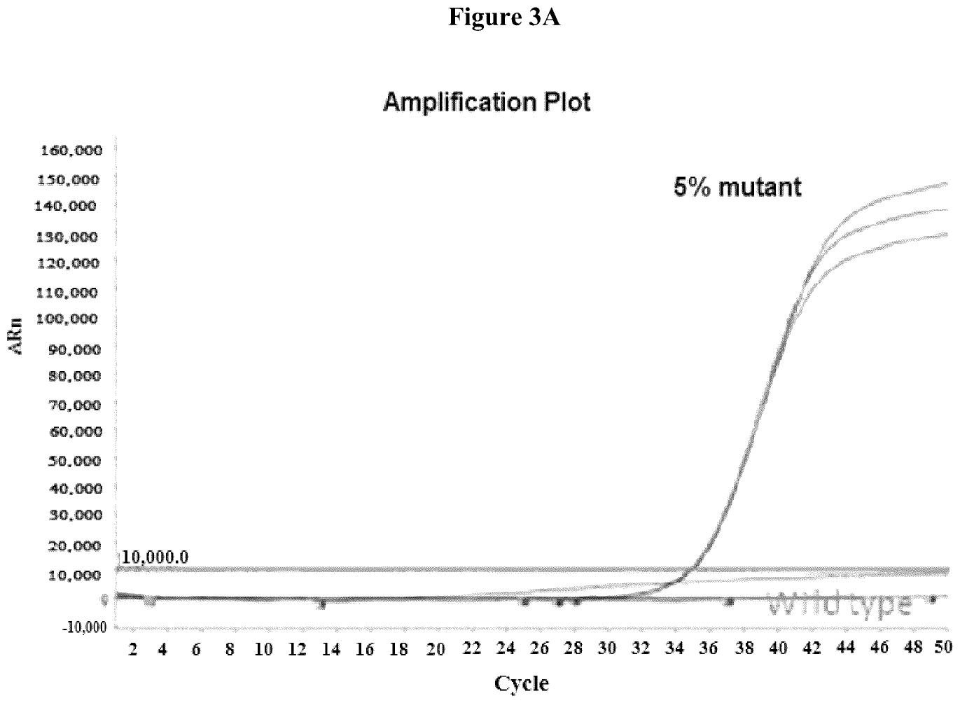

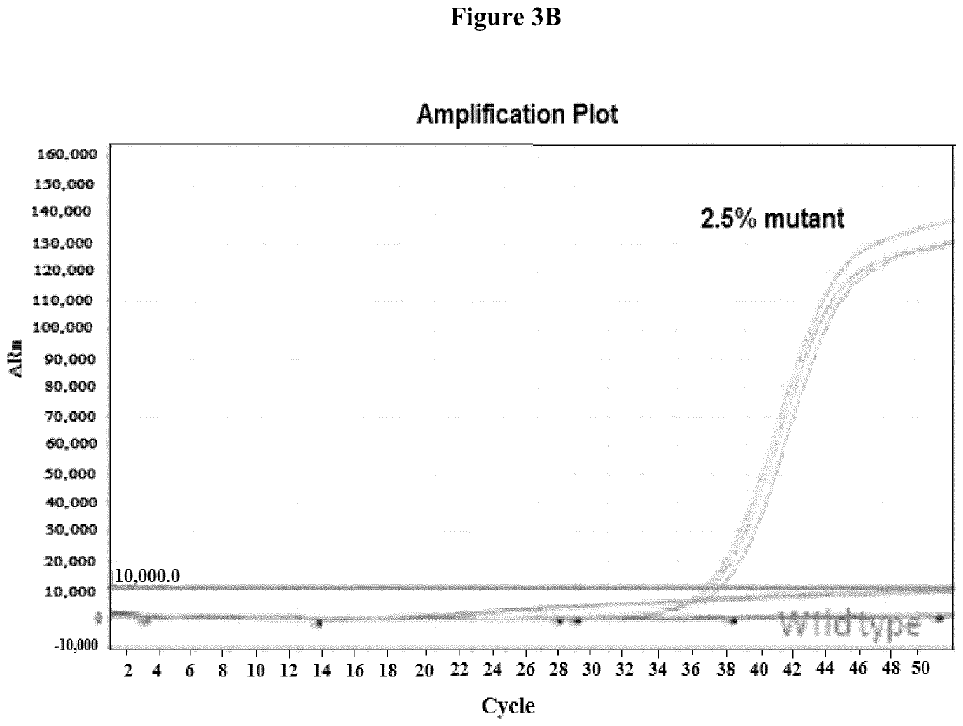

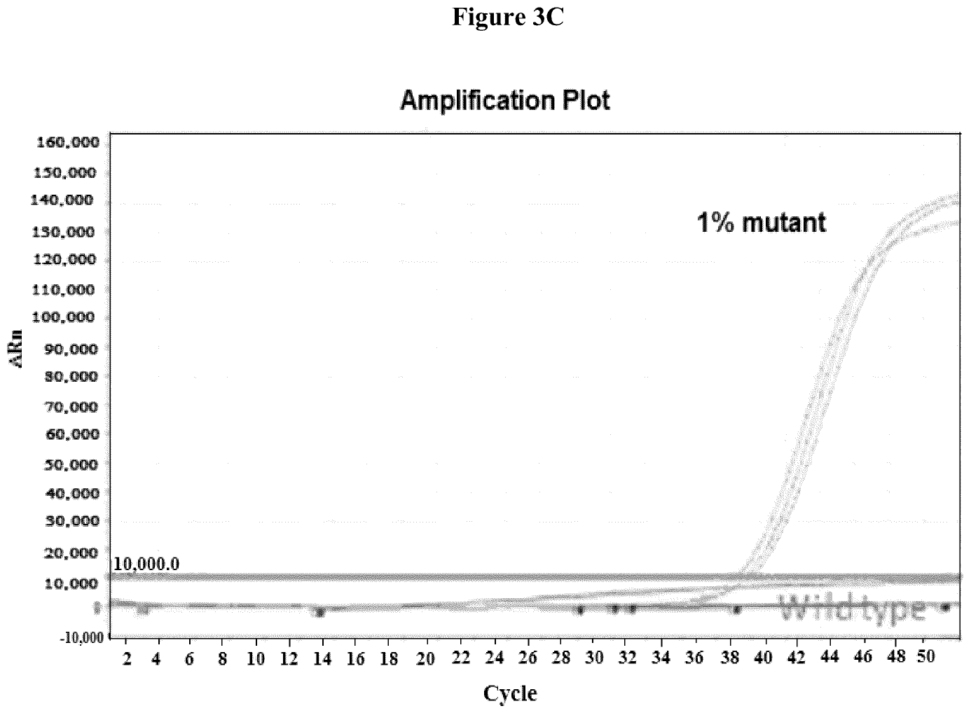

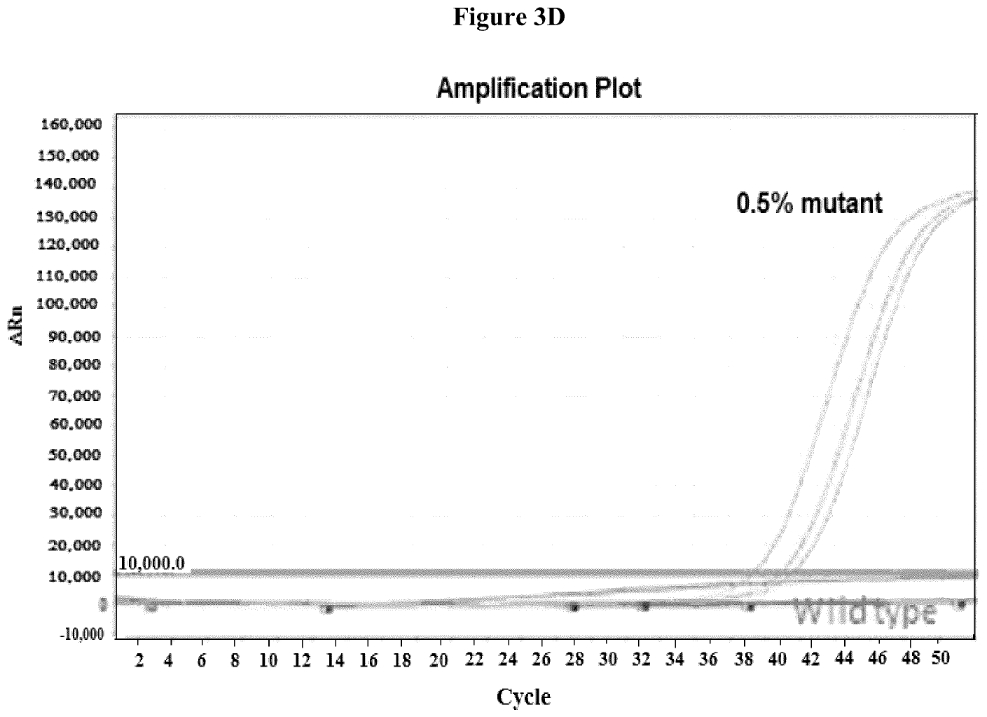

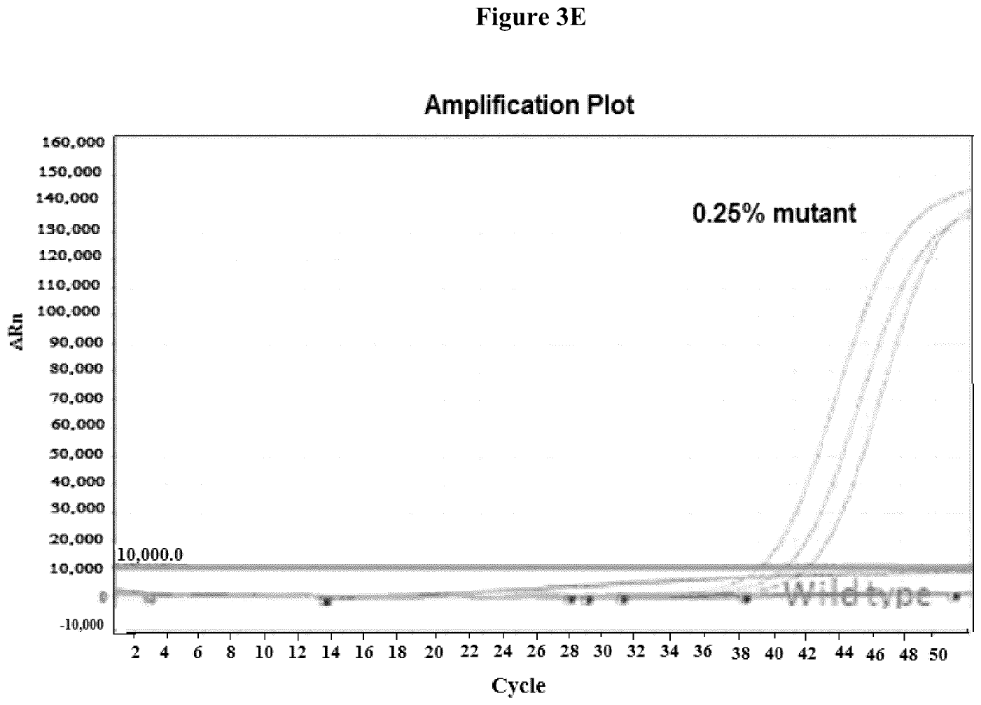

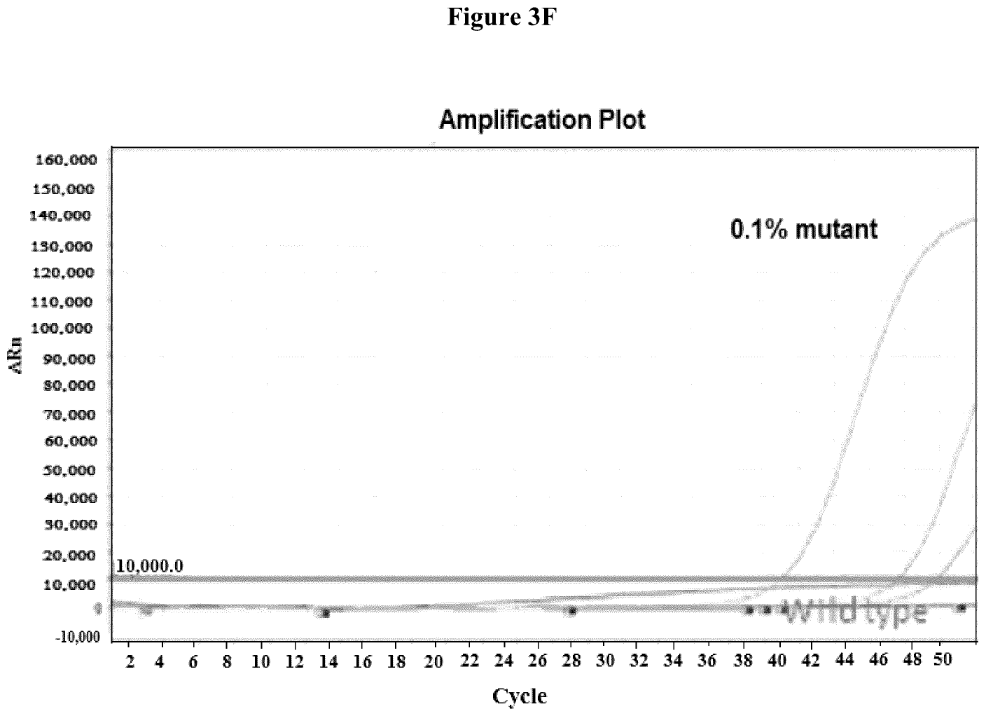

The analytical sensitivity was determined by studies involving KRAS G12C mutant genomic DNA reference samples. The known variant allele frequency (VAF) reference samples were diluted to 5%, 2.5%, 1%, 0.5%, 0.25% and 0.1% VAF respectively. The reference samples at 5ng input were evaluated. The amplification plot are shown in FIGS. 3A-F.

The tested purified reference gDNA inputs were detected at 0.25% VAF (Table 2), which is about ˜3-4 copies of mutant DNA with 5ng DNA input (1 ng gDNA about 330 genomic copies). Therefore, the limit of detection of KRAS G12C is 0.25% VAF.

TABLE 2
KRAS G12C detection sensitivity

| | Ct of KRAS G12C | Ct of ACTB |
|---|---|---|
| Mutant percentage | (Mean ± SD) | (Mean ± SD) |
| 5% | 34.9 ± 0.1 | 28.9 ± 0.2 |
| 2.5% | 35.9 ± 0.4 | 28.9 ± 0.2 |
| 1% | 37.5 ± 0.4 | 28.7 ± 0.5 |
| 0.5% | 38.5 ± 1.1 | 28.7 ± 0.1 |
| 0.25% | 39.3 ± 1.4 | 28.8 ± 0.1 |
| 0.1% | 44.1 ± 4.5 | 28.8 ± 0.3 |
| WT | 50 | 28.2 ± 0.8 |

### Analytical Specificity

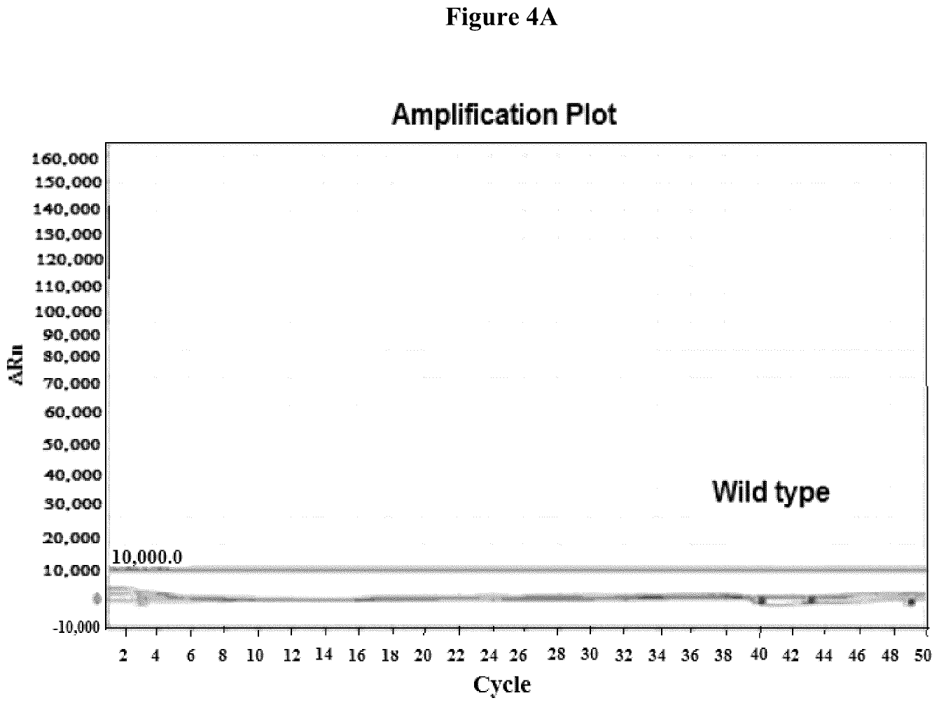

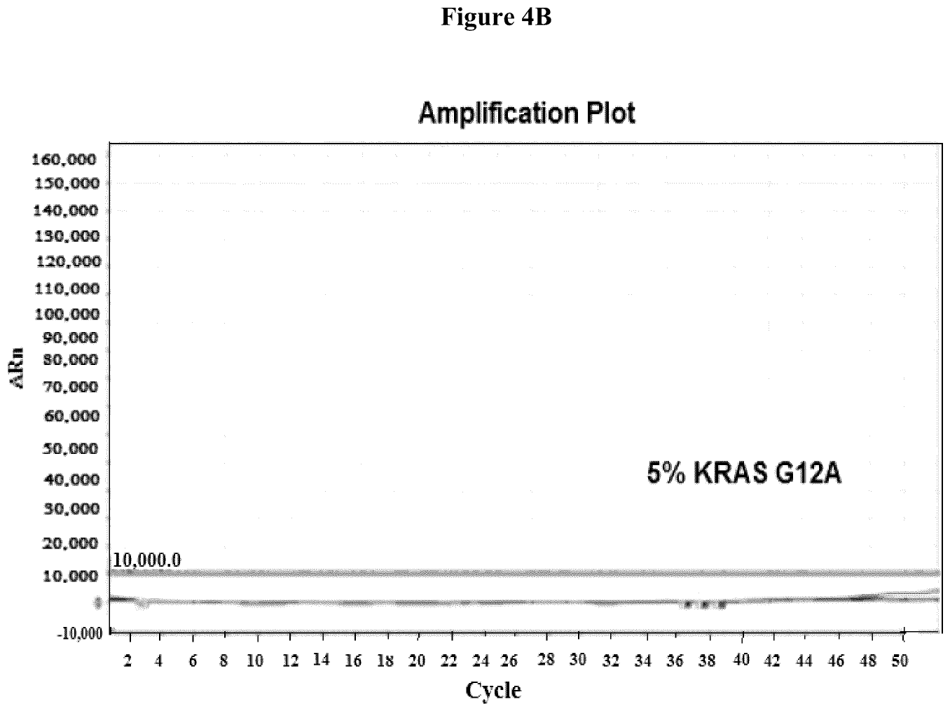

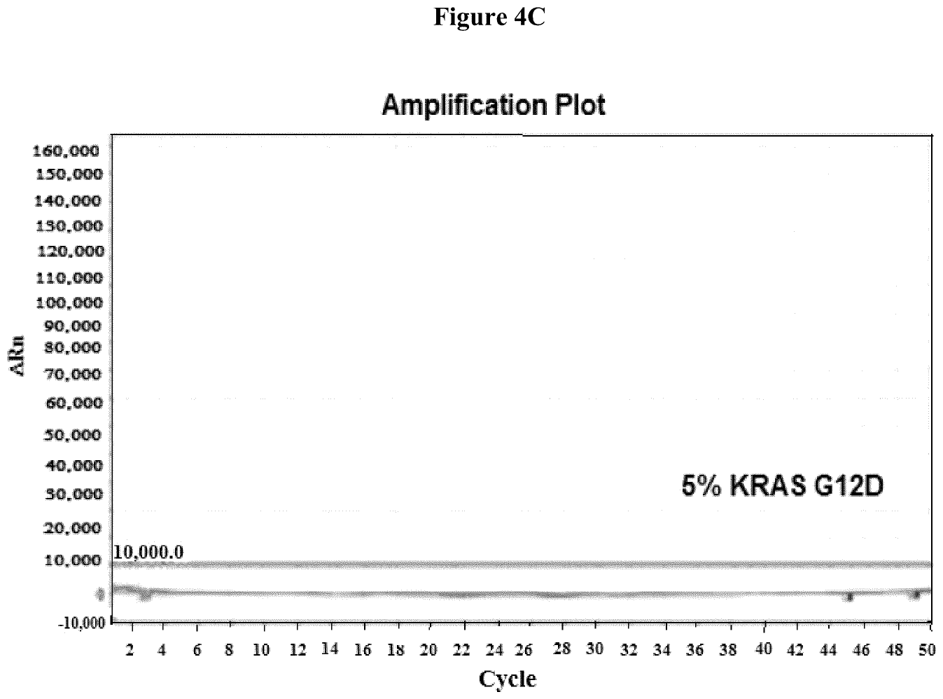

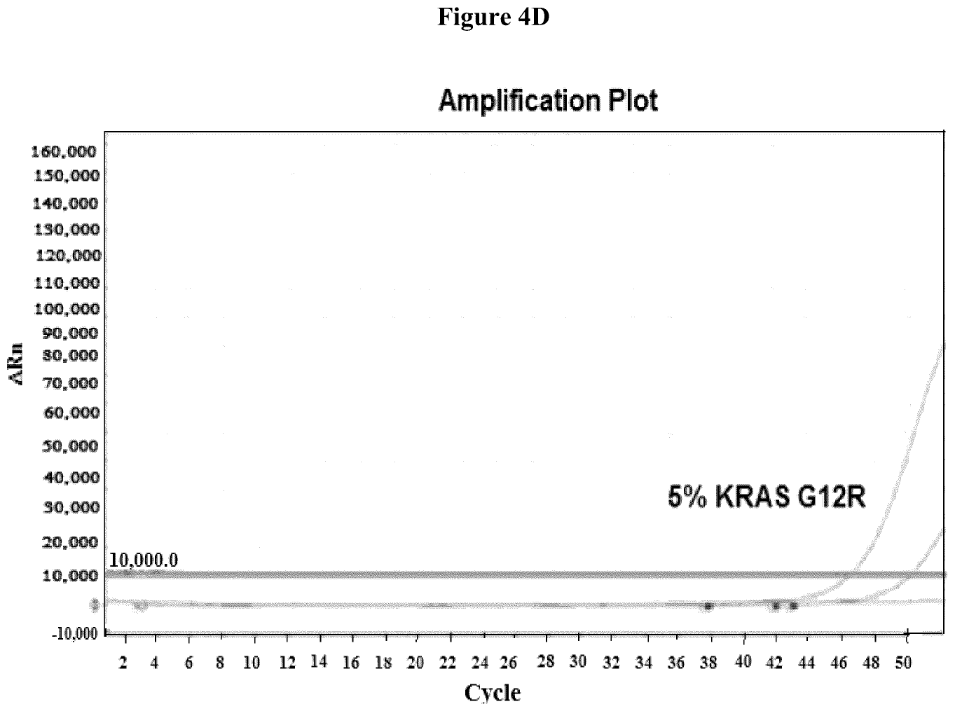

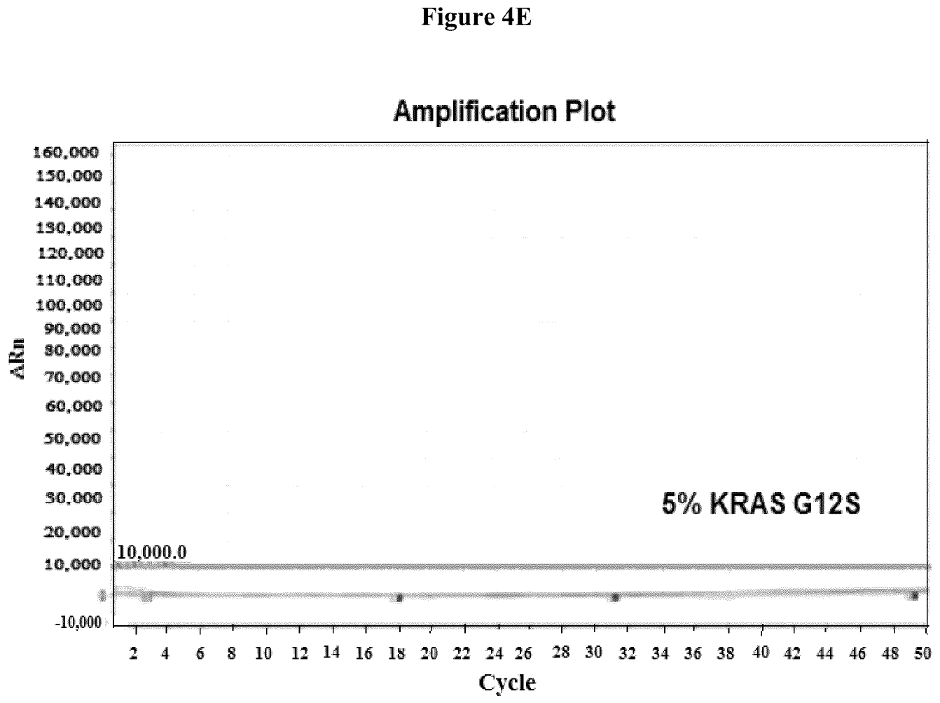

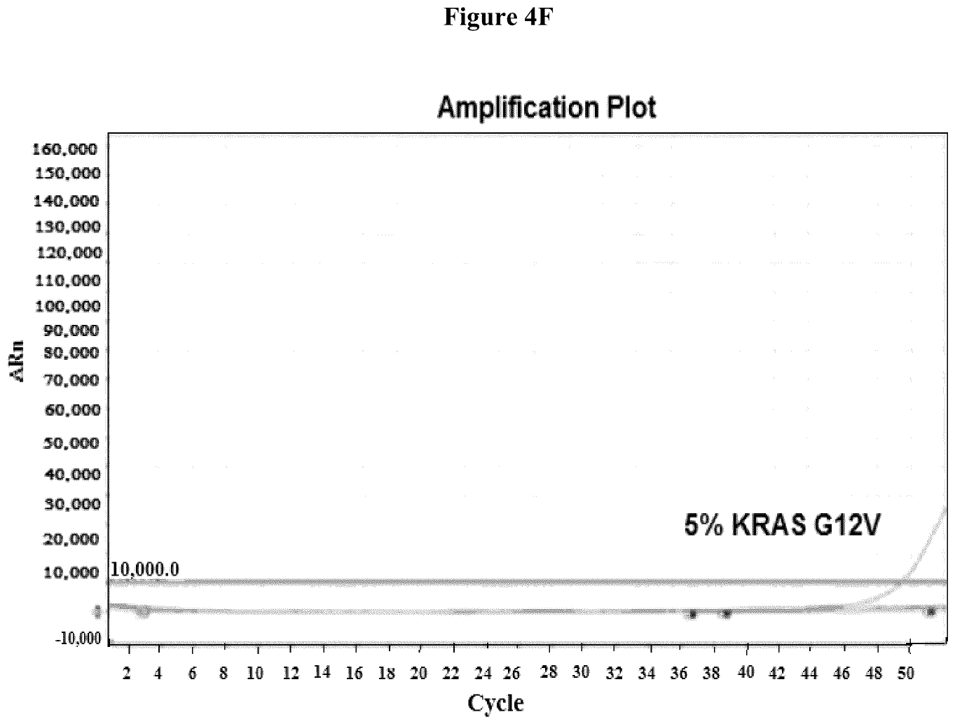

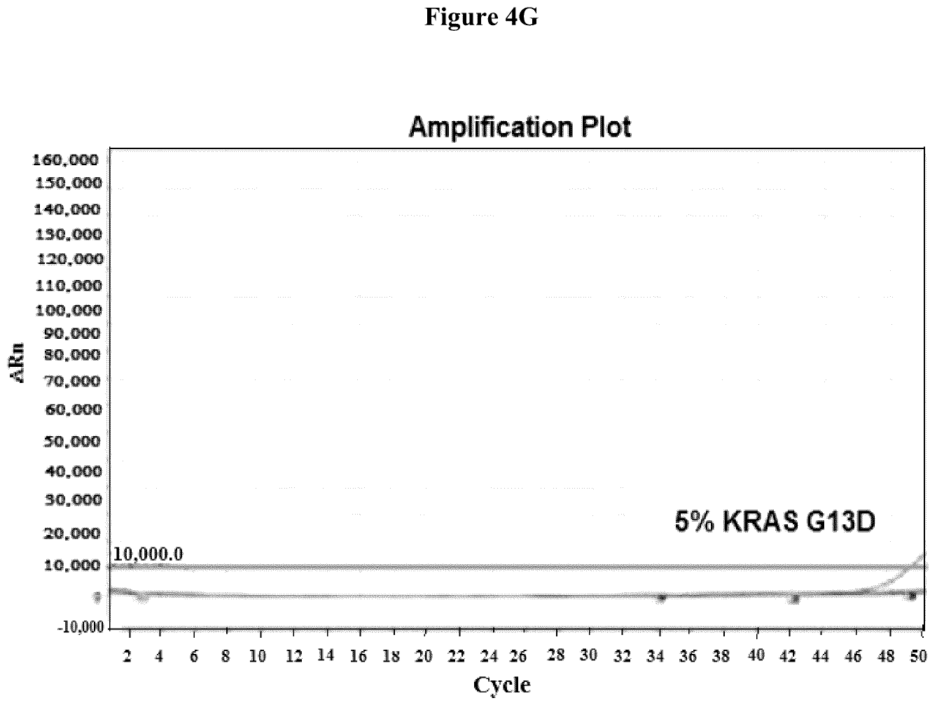

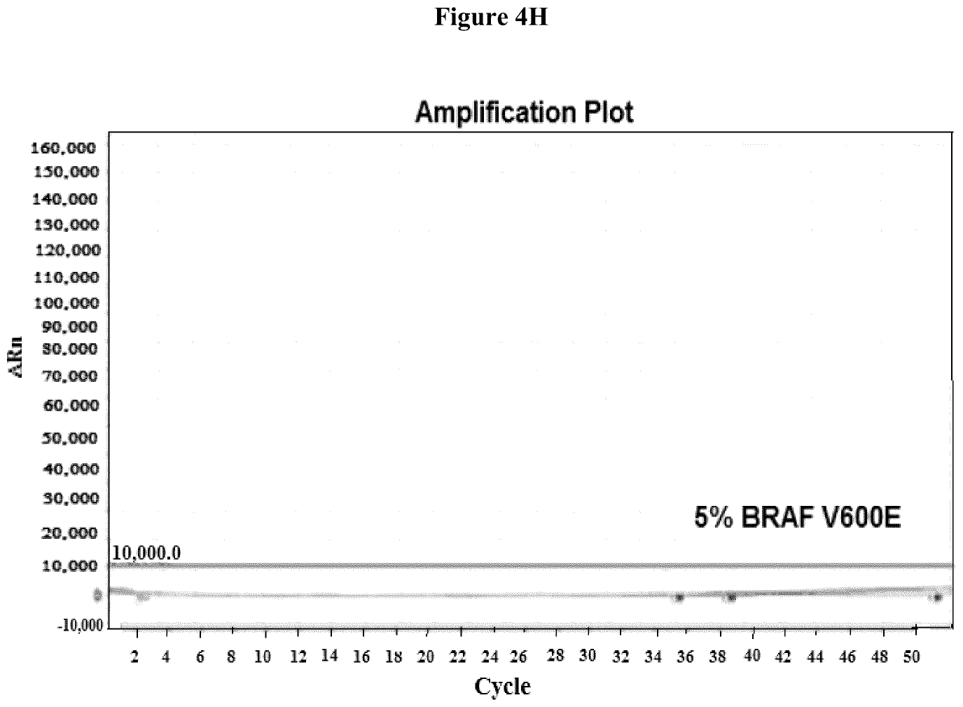

With known reference wildtype gDNA, the assay specificity is 100%. There were no false positive calls for 5 ng of gDNA per reaction (FIG. 4A). Another aspect of the assay specificity is manifested by evaluation of assay cross-reactivity. Allele specific forward primer was designed to exclusively bind to KRAS G12C mutant, but not KRAS G12A, KRAS G12D, KRAS G12R, KRAS G12S, KRAS G12V, KRAS G13D and BRAF V600E. KRAS G12A, KRAS G12D, KRAS G12R, KRAS G12S, KRAS G12V, KRAS G13D and BRAF V600E mutant reference genomic DNA at 5% VAF were used to evaluate the cross-reactivity (FIGS. 4B-H).

There is no cross-reactivity with KRAS G12A, KRAS G12D, KRAS G12S and BRAF V600E (Ct>50). Very weak signal was produced in the KRAS G12R, KRAS G12V and KRAS G13D positive samples. However, there is >10 Ct difference between the true KRAS G12C signal and the cross-talk signal from them (Table 3). Therefore, only intended target mutation of KRAS G12C can be detected by the kit.

TABLE 3
KRAS G12C detection specificity

| | Ct of KRAS G12C | Ct of ACTB |
|---|---|---|
| Mutant | (Mean ± SD) | (Mean ± SD) |
| KRAS G12C | 34.9 ± 0.3 | 28.8 ± 0.2 |
| KRAS G12A | >50 | 28.3 ± 0.3 |
| KRAS G12D | >50 | 28.9 ± 0.4 |
| KRAS G12R | 46.2 ± 2.5 | 27.5 ± 0.2 |
| KRAS G12S | >50 | 27.7 ± 0.1 |
| KRAS G12V | 47.3 | 28.4 ± 0.1 |
| KRAS G13D | 49.0 | 28.3 ± 0.2 |
| BRAF V600E | >50 | 29.0 ± 0.5 |

The KRAS G12C detection assay of the invention is shown to be a robust assay with analytical sensitivity (LOD) up to 0.25% VAF, specificity is 100% up to 5ng wild type DNA. The assay is based on xenonucleic acid (XNA) mediated PCR clamping technology and allele specific PCR. Advantages of XNA over other clamping chemistries are due to the inherent chemical properties of XNA, namely super high binding affinity to both DNA and RNA templates and higher melting temperature differentials in SNV and indels against natural DNA. XNA is thus confirmed to be a novel oligo blocker that will be applicable to a variety of cancer mutation detection assays to improve assay sensitivity.

The KRAS G12C detection assay has a robust analytical performance. This rapid, precise and sensitive molecular assay for KRAS G12C mutation detection has the following key benefits. Firstly, the assay is simple and easy to use. This singleplex qPCR assay is easy to setup and allows fast result in less than 3 hours. Secondly, the assay is efficient. Only 5 ng DNA is needed for total assay input. Usually, one 10 mL streck tube blood can yield 30ng cfDNA, which is more than enough for one testing. Most importantly, the assay is specific and sensitive. No cross-reactivity with wild-type DNA, and very low cross-reactivity with 6 other KRAS and BRAF mutations. DNA at 5ng per reaction can be routinely detected at 0.25% VAF.

In a further embodiment of the invention, there is provided a PCR kit for detecting if a patient is responsive to anti-EGFR therapy, comprising PCR reagent mixes for detection of KRAS mutations at one or more of codons 12, 13, and 61, comprising the KRAS oligonucleotides of the present invention; Taq polymerase; and instructions for use. The KRAS reagent mixes in the KRAS kit may each be individually prepared for singleplex or multiplex detection of KRAS mutations, respectively. In a singleplex format, the KRAS kit would include individual KRAS reagent mixes comprising oligonucleotides specific to each of the codon 12, 13, and 61 KRAS mutations. In a multiplex format, the individual KRAS reagent mixes may include oligonucleotides specific to two or more of the codon 12, 13, and 61 KRAS mutations. It is to be understood that the KRAS kit may include a combination of reagent mixes for singleplex and multiplex screening. For example, a KRAS kit may include reagent mixes for singleplex screening of KRAS and multiplex screening.

The invention provides a rapid, precise, and sensitive assay to enable molecular detection of KRAS G12C mutation. This XNA and allele specific qPCR-based technology can sensitively and specifically detect KRAS G12C mutation, which can be used as a potential companion diagnostic test to select potential patients for KRAS G12C inhibitor treatment.

### Example 6

The Following is exemplary of XNA Oligomer Synthesis:

### PART I. Synthetic Procedure of the Fmoc Oxy-Aza-T XNA Monomer.

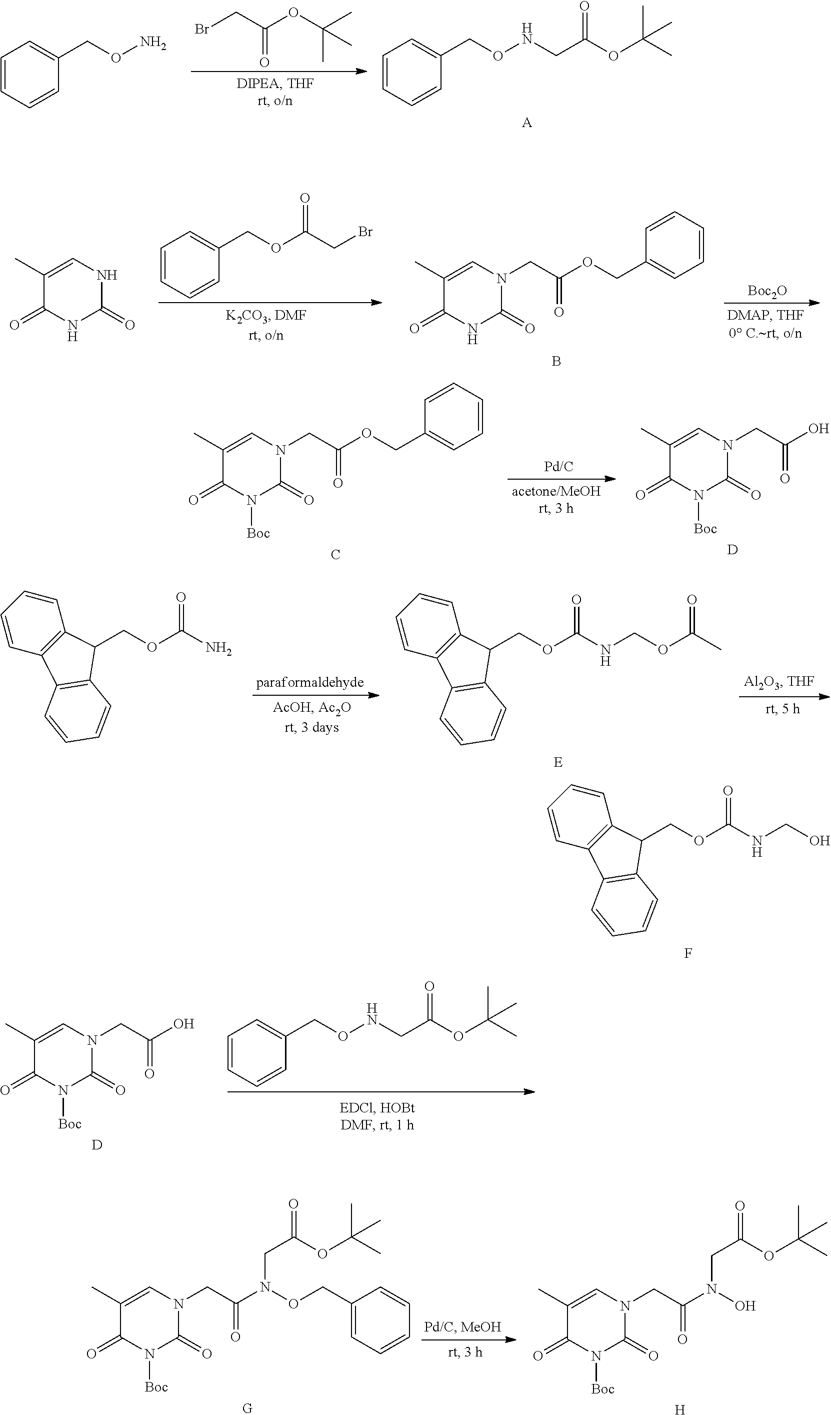

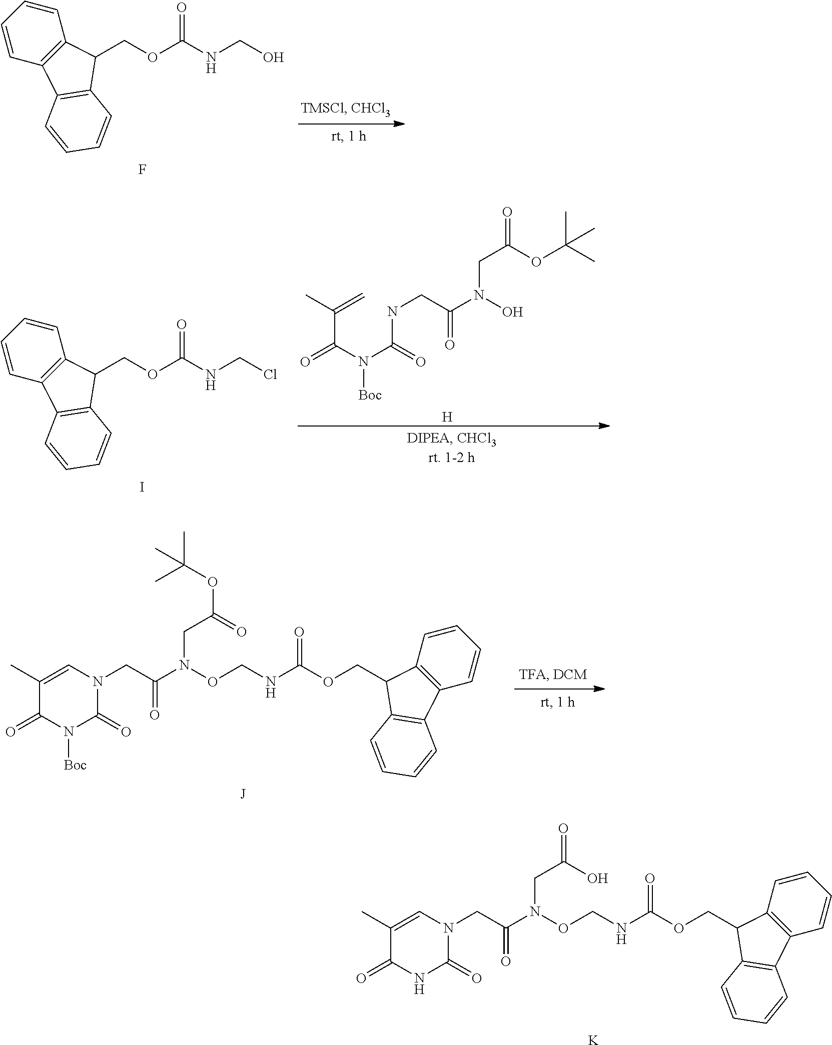

The other oxy-aza nucleotide Monomers A, C and G are prepared similarly with suitable protecting groups on the nucleoside bases.

Step 1: To a solution of O-benzylhydroxylamine (2.00 g, 15.9 mmol) and diisopropylethylamine (3.08 mL, 17.51 mmol) in THF (25 mL) was added dropwise tert-butyl 2-bromoacetate (2.5 mL, 16.71 mmol) in TRF (10 mL). The reaction mixture was stirred at 50° C. for 4 hours then at room temperature overnight. Solvent was removed under vacuum to obtain crude which was purified by Biotage Isolera flash column to obtain title compound A (1.17 g, 29.4%) as a colorless oil.

Step 2: Thymine (3.00 g, 23.0 mmol) and potassium carbonate (3.30 g, 24.0 mmol) were dissolved in dry N,N-dimethylformamide (˜70 mL). Benzyl bromoacetate (3.50 mL, 22.0 mmol) was added dropwise and the reaction mixture was stirred at room temperature overnight. The suspension was filtered and solvent was removed to obtain a residue which was purified by Biotage flash column to obtain compound B (4.09 g, 61.4%) as a white solid.

Step 3: Benzyl 2-(5-methyl-2,4-dioxo-3,4-dihydropyrimidin-1(2H)-yl)acetate (3.00 g, mmol), di-tert-butyl decarbonate (4.92 mL, 22.0 mmol), and 4-dimethylaminopyridine (2.56 g, 22 mmol) were added to TRF (˜30 mL) at 0° C. The reaction mixture was stirred at 0° C. for 30 minutes and then at room temperature overnight. The solvent was removed. The residue was dissolved in dichloromethane (100 mL) and washed with water, brine, and dried over anhydrous MgSO₄, filtered and concentrated. The crude was purified by Biotage flash column to obtain compound C (2.91 g, 71.1%) as a white solid.

Step 4: To a solution of tert-butyl 3-(2-(benzyloxy)-2-oxoethyl)-5-methyl-2,6-dioxo-2,3-dihydropyrimidine-1(6H)-carboxylate (2.91 g, 7.38 mmol) in methanol (30 mL) and acetone (30 mL), 5% Pd/C (582 mg) was added. The reaction mixture was degassed with hydrogen 3 times and stirred at room temperature under hydrogen for 3 hours. The mixture was filtered with celite and washed with methanol and acetone. The filtrate was concentrated to obtain crude compound D (1.84 g, 83.3%).

Step 5: (9H-fluoren-9-yl)methyl carbamate (3.00 g, 12.0 mmol) and paraformaldehyde (0.43 g, 14.0 mmol), were suspended in a mixture of acetic acid (22.5 mL) and acetic anhydride (70 mL). The reaction mixture was stirred at room temperature for 3 days and then filtered. The solvent was removed by distillation in vacuum and the crude was purified by flash column to get compound E (3.46 g, 85.9%) as a white solid.

Step 6:(9H-fluoren-9-yl)methoxy)carbonyl)amino)methyl acetate (3.40 g, 10.0 mmol) was dissolved in THF (˜10 mL) and loaded on a 68-gram neutral alumina column. The loaded cartridge was allowed to stand for 5 hours then eluted by THF, and thereafter concentrated to obtain compound F (1.28 g, 43.5%) as a white solid.

Step 7: N,N-diisopropylethylamine (1.15 mL, 6.49 mmol) was added to a solution of 2-(3-(tert-butoxycarbonyl)-5-methyl-2,4-dioxo-3,4-dihydropyrimidin-1(2H)-yl)acetic acid (1.03 g, 3.245 mmol), tert-butyl 2-((benzyloxy)amino)acetate (0.89 g, 3.57 mmol), 1-Ethyl-3-(3-dimethylaminopropyl)carbodiimide-HCl (3.38 g, 17.13 mmol) and hydroxybenzotriazole hydrate (2.68 g, 17.13 mmol) in N,N-dimethylformamide (˜40 mL). The reaction mixture was stirred at room temperature overnight and diluted with dichloromethane (˜50 mL). The solution was washed with water, brine, dried over anhydrous MgSO₄, filtered and concentrated. The crude was purified by flash column to obtain compound G (1.08 g, 59.5%) as a white solid.

Step 8: To a solution of tert-butyl 3-(2-((benzyloxy)(2-(tert-butoxy)-2-oxoethyl)amino)-2-oxoethyl)-5-methyl-2,6-dioxo-2,3-dihydropyrimidine-1(6H)-carboxylate (Compound G; 1.08 g, 2.04 mmol) in methanol (10 mL), 5% Pd/C (216 mg) was added. The reaction mixture was degassed with hydrogen for 3 times and stirred at room temperature under hydrogen for 3 hours. The mixture was filtered by celite and washed with methanol. The filtrate was concentrated to obtain a crude compound H (865 mg, 97.6%) as white foam.

Steps 9 and 10: To a solution of (9H-fluoren-9-yl)methyl (hydroxymethyl)carbamate (Compound F; 1.03 g, 3.63 mmol) in chloroform (40 mL), trimethylsilyl chloride (0.93 mL, 7.267 mmol) was added dropwise and stirred at room temperature for 1 hour. After 1 hour, tert-butyl 3-(2-((2-(tert-butoxy)-2-oxoethyl)(hydroxy)amino)-2-oxoethyl)-5-methyl-2,6-dioxo-2,3-dihydropyrimidine-1(6H)-carboxylate (1.74 g, 4.00 mmol) and N,N-diisopropylethylamine (2.58 mL, 14.53 mmol) were added to the above solution. The reaction mixture was stirred at room temperature for 1 hour. The reaction mixture was washed with water, brine, dried over anhydrous Na₂SO₄, filtered, and concentrated to get the residue which was purified by flash column to get compound J (762 mg, 30.0%) as a white solid.

Step 11: To a solution of tert-butyl 3-(7-(2-(tert-butoxy)-2-oxoethyl)-1-(9H-fluoren-9-yl)-3,8-dioxo-2,6-dioxa-4,7-diazanonan-9-yl)-5-methyl-2,6-dioxo-2,3-dihydropyrimidine-1(6H)-carboxylate (0.60 g, 0.857 mmol) in dichloromethane (˜12 mL), trifluoroacetic acid was added (˜5 mL, 85.8 mmol) at 0˜5° C. The reaction mixture was stirred at room temperature for 1 hour. The mixture was concentrated to obtain a residue which was purified by Biotage Isolera flash column to obtain the title compound (220 mg, 48.0%) as an off-white solid. ¹H NMR (300 MHz, CDCl3): 10.3 (s, 1H), 8.75 (s, 1H), 7.88 (d, J=7.5 Hz, 2H), 7.69 (d, J=7.3 Hz, 2H), 7.44-7.29 (m, 5H), 4.92 (d, J=6.1 Hz, 2H), 4.66 (s, 2H), 4.40-4.37 (m, 2H), 4.25 (t, J=6.4 Hz, 1H), 4.08-3.97 (m, 2H), 1.73 (s, 3H) ppm.LC-MS [M+H]⁺: 508.97, [M+Na]⁺: 531.23. HPLC purity: 95.7% at 254 nm.

PART II. Synthesis of Chemically-Modified EGFR c797S XNA, Using Fmoc Oxy-Aza-T XNA Monomer (Bold Red) to Replace the Regular Fmoc-T Monomer (Bold Black) as Specified Below:
Egfr c797S
Regular-T original sequence:

SEQ ID NO: 21
5′-D-LYS-O-TTCGGCTGCCTCCTGG-3′

### Partial Oxy-Aza-T Replacement Sequence:

SEQ ID NO: 22
5′-D-LYS-O-TTCGGCT_{OA}GCCT_{OA}CCTGG-3′

where OA is oxy-aza.

### a) Solid-Phase Synthesis Step

This step has been conducted on an INTAVIS MultiPep automatic synthesizer (INTAVIS Bioanalytical Instruments AG, Cologne, Germany), coupled with a compact Welch vacuum pump (4 m³ per hour ventilation rate), a 20-liter stainless steel waste container, and a long ventilation hose to discharge the solvent vapor and smell from the system into a nearby chemical fume hood.

In a typical 24-port (4×6) array plate, a micro column (0.5-ml capacity) with PTFE filters was inserted tightly into a chosen port. A certain weight of TentaGel resin (1 micromole, namely 10.0 mg resin at 0.10 mmol/gram loading capacity) was loaded to this column.

Four regular monomers (Fmoc-T/A/C/G) and O-linker monomer (Fmoc-AEEA-OH) were purchased commercially (98+% purity) and prepared freshly as 0.3 M solutions in N-methyl 2-pyrrolidone (NMP); Fmoc-D-Lysine(t-Boc) monomer as a 0.5 M solution in NMP. This unconventional Fmoc Oxy-Aza-T monomer was also made as a 0.3 M solution in a smaller 15-ml polypropylene vial (100 mg about 0.2 mmol dissolved in 600 uL of NMP solvent), and was accordingly given a new code of monomer in the program (perhaps like “oaT” ?). All other reagents (from Sigma-Aldrich if not specified otherwise, with purity of 98% or higher) include 0.5 M DMF solution of HATU (from P3 BioSyetems Inc, 1-[Bis(dimethylamino)-methylene]-1H-1,2,3-triazolo[4,5-b]pyridinium 3-oxid hexafluorophosphate, Hexafluoro-phosphate Azabenzotriazole Tetramethyl Uronium) for carboxy activation, a base solution containing 1.2 M DIPEA and 1.8 M 2,6-lutidine (1:1, v/v) in DMF for acid scavenger, a 20% piperidine solution in DMF (v %) for Fmoc group deprotection, 5% (v %) acetic anhydride in DMF for amino capping procedure, NMP and methylene chloride and ethanol for column wash use.

After the preparative procedures above are completed, the XNA sequence was input to the operating PC's INTAVIS program with double check. The automatic synthesis on the TentaGel resin was started from the 3′ terminal of XNA sequence (namely from C-terminal) following this program, using a pre-set 1-micromole-scale double-coupling synthesis method. Briefly, in a typical cycle, a double deprotection, a double coupling and a single capping procedure was included to assure the sufficiently high-yielding and clean synthesis; a molar ratio of HATU/Base/monomer/amino=5:25:5:1 was chosen in general. The synthesizer was programmed to automatically repeat the cycles from 3′ end to 5′ end, till the 5′ end of the sequence that is the D-lysine terminus here. At this last cycle, the resin was thoroughly washed and then dried. Resin weight was found to increase obviously.

### b) Resin Cleavage and Side-Chain Deprotection

The dried resin was transferred to a 50-ml polypropylene centrifuge vial, using methylene chloride as the suspension medium for an easy and complete transfer, then dried in vacuum. A cocktail of TFA/m-cresol/TIPS/water (90:5:2.5:2.5, v %) was added (1000 uL for 1 umol resin), the cleavage/deprotection procedure was carried out at room temperature on an orbital shaker for 3 hrs at 160 cpm. The resin was then filtered out, the filtrate (˜1 mL) was mixed with 40-mL of cold anhydrous ether (0-5 Celsius degree), a significant amount of off-white loose precipitate appeared. The precipitate was collected and vacuum-dried after high-speed centrifuge (4500 cpm, 20 minutes) on a WAVERLY fixed-angle centrifuge. The crude solid was redissolved in about 300 ul of water for HPLC purification.

### c) HPLC Purification of Fmoc-ON XNA

Our Agilent HPLC 1100 system consists of a G1322A degasser, G1311A Quaternary Pump, G1313A automatic sampler, G1316A column compartment with temperature control and G1315B diode array detector.

A typical HPLC purification run is conducted as below on a Aeris peptide XB-C18 RP-HPLC column (100×4.5 mm, 3.6 um particle size): 5%-29% gradient of mobile phase B in 0-28 minutes (mobile phase A: 0.1% TFA in water; mobile phase B: 0.1% TFA in acetonitrile) for elation of the XNA product and byproduct peaks, followed by 29%-60% wash for 4 minutes (28-32 min), and then 60%-5% wash back to equilibrate the column for the next run (32-36 min). Other parameters: 1.0 ml/min flow rate, column temperature 50.0+/−0.5 Celsius degree, UV detection at 260 nm and 205 nm simultaneously (detecting DNA base and TFA impurity respectively), a single sample injection as 100 ul each run.

The XNA product peak fractions (a main and sharp peak usually in the range of 17-23 min) were collected and combined, as the eluted solution of purified XNA (Fmoc-ON version).

### d) Lyophilization of Fmoc-ON XNA

The purified Fmoc-ON XNA solution (in mixed solvent of water and acetonitrile, with 0.1% TFA) was transferred to a 50-ml centrifuge vial (polypropylene) and frozen either in cold bath of dry-ice/acetone or −80 Celsius degree freezer, then subjected to lyophilization.

A 1200-ml LABCONCO flask including the frozen sample vial(s) was attached to a port of multifold of a LABCONCO desktop lyophilizer (Freezone 4.5 model) which was already stabilized at −40 Celsius degree and approximately 100 microbar (0.1 mmHg). The process continued usually for 8-48 hours depending on total sample volume. Upon completion of this process, a loose and white solid was obtained as the dried XNA product (Fmoc-ON version).

This version of purified XNA can be used directly after being re-dissolved in water or TE buffer. The product quantity can be calculated by the base concentration measured at 260 nm and the XNA solution total volume, then the synthetic yield (%) can be calculated. MALDI-TOF mass spectrum of the synthesized XNA (Fmoc-ON version) was measured on Shimadzu Axima MALDI-TOF mass spectrometer and data was recorded, using sinapinic acid as the matrix and the bovine cytochrome C protein as the molecular weight reference standard. If even higher water solubility is mandatory, then the deprotection of the terminal Fmoc group of the purified XNA above can be further processed, see Step (e) and Step (f) below.

### e) Additional D-Lysine Fmoc Deprotection and Further HPLC Purification

The purified XNA above is redissolved in small amount of DMF (e.g. 300 ul for each micromole), then a calculated amount of piperidine was added in at room temperature so as to make it a 10% piperidine/DMF solution, the deprotection only took a few minutes to complete. Following the deprotection, 40-ml of cold anhydrous ether is added to precipitate the crude product.

Another round of HPLC was repeated with the conditions listed above, the Fmoc-OFF XNA peak comes out earlier, usually in the range of 10-15 min window due to its increased hydrophilicity and thus less stronger adsorption on the RP-HPLC column. All product fractions were collected and combined.

### f) Further Lyophilization and Formulation

Lyophilization procedure is similar to the procedure (d) described above, during which the acetonitrile and TFA can be completely removed, leaving a final powder product of XNA (Fmoc-OFF version).

The product quantity can be calculated by the base concentration measured at 260 nm and the XNA solution total volume, and then the synthetic yield (%) can be calculated.

MALDI-TOF mass spectrum of the synthesized XNA (Fmoc-OFF version) was measured on Shimadzu Axima MALDI-TOF mass spectrometer and data was recorded, using sinapinic acid as the matrix and the bovine cytochrome C protein as the molecular weight reference standard.

The powder XNA is then redissolved in either pure water or TE buffer, as an aqueous solution of typically 200 micromolar concentration. The resulting solution can be either directly used for the subsequent XNA clamping-based qPCR or aliquoted (e.g. 50 ul=10 nmol) for lyophilization again to store for long term.

Other XNA oligomers can be synthesized in a similar fashion composed partially or entirely of oxy-aza, aza-aza and/or sulfa-aza (thio-aza) XNA monomers.

Other XNA sequences used in the invention and more in particular with respect to Example 6 of the invention includes:

EGFR G719
SEQ ID NO: 8
D-Lys-O-CG_{OA}GA_{AA}GCCC_{OA}AGCACTTTGAT
EGFR Ex 19Del
SEQ ID NO: 9
D-Lys-O-C_{OA}G_{OA}G_{OA}A_{OA}G_{OA}ATGTTGCT_{OA}T_{OA}CTCTTAATTCC
EGFR T790
SEQ ID NO: 10
D-Lys-O-T_{AA}C_{AA}A_{AA}TCAC_{OA}GC_{OA}AGCTC
EGFR L858
SEQ ID NO: 11
D-Lys-O-GGCCAGC_{OA}C_{OA}CAAAAT_{AA}CTGT
NRAS G12
SEQ ID NO: 12
D-Lys-O-C_{OA}AA_{OA}CAC_{AA}C_{AA}AC_{OA}CTGCTCCAACCACCAC
NRAS A59
SEQ ID NO: 13
D-Lys-O-TTC_{OA}TTGTC_{OA}CA_{OA}GCT_{AA}GTAT_{AA}CCAGTATG
KRAS G12
SEQ ID NO: 14
D-Lys-O-C_{AA}TACGCCACC_{OA}AGCTC_{OA}CAACTACCA
KRAS A59
SEQ ID NO: 15
D-Lys-O-C_{OA}TCTTGACCT_{OA}GCT_{OA}GTGT_{AA}CGAG
KRAS A146
SEQ ID NO: 16
D-Lys-O-T_{OA}GTCTTT_{AA}GCTG_{OA}ATGT
APC E1309
SEQ ID NO: 17
D-Lys-O-C_{AA}TGAC_{OA}CTAGT_{OA}TCCAAT_{AA}CTTTTCTT
PIK3CA H1047
SEQ ID NO: 18
D-Lys-O-A_{OA}ATGAT_{AA}GCACATCAT_{OA}GGTGGCTG
CTNNB1 S45
SEQ ID NO: 19
D-Lys-O-C_{AA}TCCTT_{OA}CTCT_{AA}GAG_{OA}TG
BRAF V600
SEQ ID NO: 20
D-Lys-O-A_{OA}TC_{OA}GAGAT_{AA}TT_{OA}CACT_{AA}GTAGCTAGAC

In sequences 8 through 20 the subscripts designations OA and AA stand for oxy-aza and aza-aza moieties in the Xenonucleic acid.

### REFERENCES

[1] J. Downward, “Targeting RAS signalling pathways in cancer therapy,” Nature Reviews Cancer. 2003.
[2] J. M. L. Ostrem and K. M. Shokat, “Direct small-molecule inhibitors of KRAS: From structural insights to mechanism-based design,” Nature Reviews Drug Discovery. 2016.
[3] S. M. Sweeney et al., “AACR project genie: Powering precision medicine through an international consortium,” Cancer Discov., 2017.
[4] Amgen, “Q2 2019 pipeline, Amgen.,” 2019. [Online]. Available: https://www.amgenpipeline.com/˜/media/amgen/full/www-amgenpipeline-com/charts/amgen-pipeline-chart.ashx.
[5] E. M. Kyger, M. D. Krevolin, and M. J. Powell, “Detection of the hereditary hemochromatosis gene mutation by real-time fluorescence polymerase chain reaction and peptide nucleic acid clamping,” Anal. Biochem., vol. 260, no. 2, pp. 142-148, July 1998.
[6] D. Jeong et al., “Rapid and sensitive detection of KRAS mutation by peptide nucleic acid-based real-time pcr clamping: A comparison with direct sequencing between fresh tissue and formalin-fixed and paraffin embedded tissue of colorectal cancer,” Korean J. Pathol., vol. 45, no. 2, pp. 151-159, April 2011.
[7] M. Beau-Faller et al., “Detection of K-Ras mutations in tumour samples of patients with non-small cell lung cancer using PNA-mediated PCR clamping,” Br. J. Cancer, vol. 100, no. 6, pp. 985-992, March 2009.
[8] P.-Y. Kwok and X. Chen, “Detection of Single Nucleotide Polymorphisms,” 2003.
[9] C. R. Newton et al., “Analysis of any point mutation in DNA. The amplification refractory mutation system (ARMS),” Nucleic Acids Res., vol. 17, no. 7, pp. 2503-2516, April 1989.
[10] J. Liu et al., “An improved allele-specific PCR primer design method for SNP marker analysis and its application,” Plant Methods, vol. 8, no. 1, p. 34, August 2012.

All literature and similar materials cited in this application including, but not limited to, patents, patent applications, articles, books, treatises, and internet web pages, regardless of the format of such literature and similar materials, are expressly incorporated by reference in their entirety for any purpose as if they were entirely denoted. In the event that one or more of the incorporated literature and similar materials defines or uses a term in such a way that it contradicts that term's definition in this application, this application controls.

Although the foregoing description contains many specifics, these should not be construed as limiting the scope of the present invention, but merely as providing illustrations of some of the presently preferred embodiments. Similarly, other embodiments may be devised without departing from the spirit or scope of the present invention. Features from different embodiments may be employed in combination. The scope of the invention is, therefore, indicated and limited only by the appended claims and their legal equivalents rather than by the foregoing description. All additions, deletions and modifications to the invention as disclosed herein which fall within the meaning and scope of the claims are to be embraced thereby.

What is claimed is:

1. A method of detecting the KRAS G12C mutations at one or more of codons; said method comprising: (a) providing a biological sample; (b) isolating DNA from said biological sample; said DNA including said KRAS G12C mutations; (c) providing a first primer probe which is allele specific and a second primer probe; wherein said first and second primer probes are targeted to said KRAS G12C mutations and wherein said primer probes allow formation of a PCR product; (d) providing a target specific xenonucleic acid clamp oligomer probe specific for a wildtype polynucleotide sequence; so that during the qPCR process only mutant templates are amplified; (e) admixing the primer probes and the xenonucleic acid clamping probe with the target nucleic acid sample; (f) performing a PCR amplification process in a reaction solution under hybridization conditions thereby generating multiple amplicons; (g) detecting said amplicons; and wherein said xenonucleic acid clamps have aza-aza, thio-aza and oxy-aza chemical functionality and selected from the group consisting of the following chemical structures: 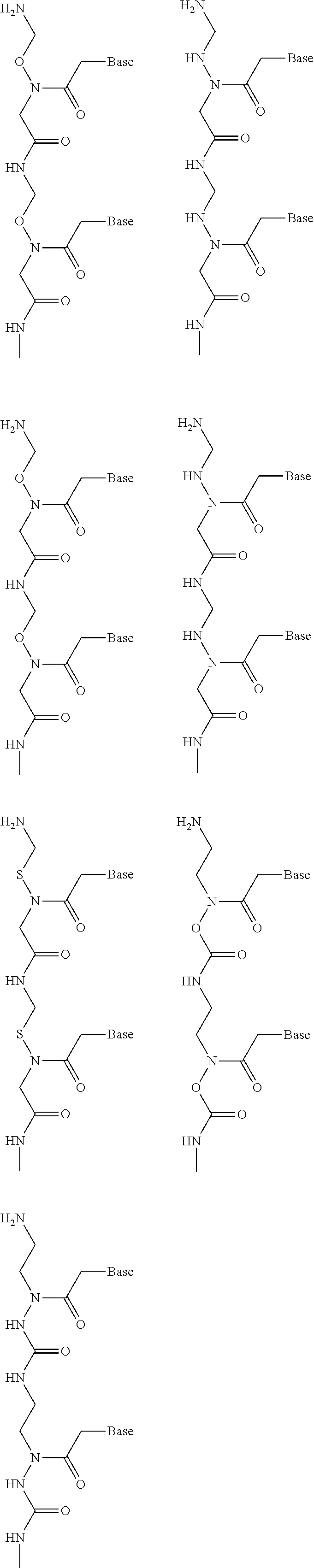
where base is selected from the group consisting of adenine, cytosine, guanine, thymine and uracil.

2. The method of claim 1, wherein the biological sample is obtained from a human or animal subject diagnosed with cancer.

3. The method of claim 1, wherein the biological sample is selected from the group consisting of formalin-fixed paraffin embedded (FFPE) tissue, fresh frozen tumor specific tissue, circulating tumor cells, circulating cell-associated DNA from plasma, and circulating non-cell associated DNA from plasma.

4. The method of claim 1, wherein the biological sample is obtained from a healthy human or animal subject.

5. The method of claim 4, wherein the biological sample is selected from the group consisting of formalin-fixed paraffin embedded (FFPE) tissue, circulating cell-associated DNA from plasma, and circulating non-cell associated DNA from plasma.

6. A method for detecting the KRAS G12C mutations at one or more of codons, said method comprising the steps of: (a) extracting DNA from a biological sample; (b) assaying the DNA via PCR for KRAS G12C mutations at one or more of codons with at least one set of oligonucleotides, wherein the at least one set of oligonucleotides comprises an allele specific forward primer, a reverse primer, a probe and a xenonucleic acid clamp to block amplification of wild type DNA and wherein said xenonucleic acid clamps have aza-aza, thio-aza and oxy-aza chemical functionality and selected from the group consisting of the following chemical structures: 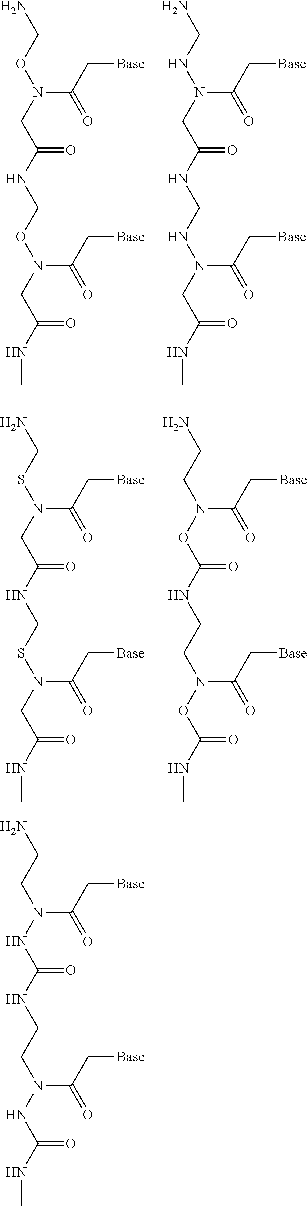
where base is selected from the group consisting of adenine, cytosine, guanine, thymine and uracil.


SEQUENCE LISTING (22 sequences)

SEQ ID NO: 1 - DNA, 29 bp, synthetic construct
  source 1..29: mol_type = other DNA
tgaatataaacttgtggtagttggagcat

SEQ ID NO: 2 - DNA, 28 bp, synthetic construct
  source 1..28: mol_type = other DNA
cctctattgttggatcatattcgtccac

SEQ ID NO: 3 - DNA, 29 bp, synthetic construct
  source 1..29: mol_type = other DNA
tctgaattagctgtatcgtcaaggcactc

SEQ ID NO: 4 - DNA, 24 bp, synthetic construct
  source 1..24: mol_type = other DNA
  misc_feature 1..24: note = D-Lysine modified
ctacgccaccagctccaactacca

SEQ ID NO: 5 - DNA, 24 bp, synthetic construct
  source 1..24: mol_type = other DNA
tctgccttacagatcatgtttgag

SEQ ID NO: 6 - DNA, 20 bp, synthetic construct
  source 1..20: mol_type = other DNA
ccagaggcgtacagggatag

SEQ ID NO: 7 - DNA, 25 bp, synthetic construct
  source 1..25: mol_type = other DNA
ccatgtacgttgctatccaggctgt

SEQ ID NO: 8 - DNA, 19 bp, synthetic construct
  source 1..19: mol_type = other DNA
  misc_feature 1..19: note = D-Lysine, oxy-aza and aza-aza modified
cggagcccagcactttgat

SEQ ID NO: 9 - DNA, 25 bp, synthetic construct
  source 1..25: mol_type = other DNA
  misc_feature 1..25: note = D-Lysine, oxy-aza and aza-aza modified
cggagatgttgcttctcttaattcc

SEQ ID NO: 10 - DNA, 14 bp, synthetic construct
  source 1..14: mol_type = other DNA
  misc_feature 1..14: note = D-Lysine, oxy-aza and aza-aza modified
tcatcacgcagctc

SEQ ID NO: 11 - DNA, 18 bp, synthetic construct
  source 1..18: mol_type = other DNA
  misc_feature 1..18: note = D-Lysine, oxy-aza and aza-aza modified
ggccagcccaaaatctgt

SEQ ID NO: 12 - DNA, 25 bp, synthetic construct
  source 1..25: mol_type = other DNA
  misc_feature 1..25: note = D-Lysine, oxy-aza and aza-aza modified
caacaccacctgctccaaccaccac

SEQ ID NO: 13 - DNA, 25 bp, synthetic construct
  source 1..25: mol_type = other DNA
  misc_feature 1..25: note = D-Lysine, oxy-aza and aza-aza modified
ttcttgtccagctgtatccagtatg

SEQ ID NO: 14 - DNA, 24 bp, synthetic construct
  source 1..24: mol_type = other DNA
  misc_feature 1..24: note = D-Lysine, oxy-aza and aza-aza modified
ctacgccaccagctccaactacca

SEQ ID NO: 15 - DNA, 21 bp, synthetic construct
  source 1..21: mol_type = other DNA
  misc_feature 1..21: note = D-Lysine, oxy-aza and aza-aza modified
ctcttgacctgctgtgtcgag

SEQ ID NO: 16 - DNA, 15 bp, synthetic construct
  source 1..15: mol_type = other DNA
  misc_feature 1..15: note = D-Lysine, oxy-aza and aza-aza modified
tgtctttgctgatgt

SEQ ID NO: 17 - DNA, 24 bp, synthetic construct
  source 1..24: mol_type = other DNA
  misc_feature 1..24: note = D-Lysine, oxy-aza and aza-aza modified
ctgacctagttccaatcttttctt

SEQ ID NO: 18 - DNA, 23 bp, synthetic construct
  source 1..23: mol_type = other DNA
  misc_feature 1..23: note = D-Lysine, oxy-aza and aza-aza modified
aatgatgcacatcatggtggctg

SEQ ID NO: 19 - DNA, 15 bp, synthetic construct
  source 1..15: mol_type = other DNA
  misc_feature 1..15: note = D-Lysine, oxy-aza and aza-aza modified
ctccttctctgagtg

SEQ ID NO: 20 - DNA, 24 bp, synthetic construct
  source 1..24: mol_type = other DNA
  misc_feature 1..24: note = D-Lysine, oxy-aza and aza-aza modified
atcgagatttcactgtagctagac

SEQ ID NO: 21 - DNA, 16 bp, synthetic construct
  source 1..16: mol_type = other DNA
  misc_feature 1..16: note = D-Lysine modified
ttcggctgcctcctgg

SEQ ID NO: 22 - DNA, 16 bp, synthetic construct
  source 1..16: mol_type = other DNA
  misc_feature 1..16: note = D-Lysine, oxy-aza and aza-aza modified
ttcggctgcctcctgg